(19) United States
(12) Patent Application Publication
(10) Pub. No.: US 2007/0275918 A1
(43) Pub. Date: Nov. 29, 2007

(54) Induction of Cellular Senescence by Cdk4 Disruption for Tumor Suppression and Regression

(75) Inventors: Hiroaki Kiyokawa, Chicago, IL (US); Xianghong Zou, Columbus, OH (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: The Board of Trustees of the University of Illinois, Urbana, IL (US)

(21) Appl. No.: 10/577,591

(22) PCT Filed: May 12, 2004
(86) PCT No.: PCT/US04/14737

§ 371 Date: Feb. 9, 2007

Related U.S. Application Data
(60) Provisional application No. 60/518,223, filed on Nov. 7, 2003.
(60) Provisional application No. 60/519,120, filed on Nov. 12, 2003.

(51) Int. Cl. A61K 31/7105; C07H 21/04; C12N 15/11

(52) U.S. Cl. 514/44; 435/375; 536/23.1

(57) ABSTRACT

The invention provides methods of inhibiting growth of tumor cells comprising contacting the cells with a Cdk4 inhibitor. The invention also provides methods of treating patients having, suspected of having, or at a high risk for developing, a cancer, comprising treatment with a Cdk4 inhibitor. The invention also relates to pharmaceutical compositions for treating such patients, wherein the pharmaceutical compositions comprise a Cdk4 inhibitor. The invention further relates to Cdk4 siRNA molecules capable of inhibiting Cdk4 expression or activity.

### FIELD OF THE INVENTION

The invention relates to methods of inhibiting growth of tumor cells. In particular, the invention relates to methods of inhibiting tumor cell growth by inhibiting expression or activity of Cdk4. The invention specifically relates to inhibiting tumor cell growth by contacting tumor cells with a Cdk4 inhibitor. The invention also relates to methods of treating an animal, particularly a human patient having, suspected of having, or at a high risk for developing, cancer or growing tumor cells. The invention also relates to pharmaceutical compositions of such Cdk4 inhibitors useful for treating such patients.

### BACKGROUND OF THE INVENTION

Cell growth is a regulated process conventionally described as the cell cycle, comprising then phases G1 (1^{st} growth phase), S (DNA synthesis), G2 (2^{nd} growth phase) and M (mitosis) (Lewin, 2000, GENES VII, Oxford University Press, Oxford). A balance of growth-stimulatory and inhibitory signals regulates G1 progression of the cell cycle, as well as the transition between proliferation and quiescence (termed the G0 phase) (Pardee, 1989, Science 246:603-608). Perturbed control of the G1 phase of the cell cycle is a critical step for cellular transformation and tumorigenesis (Hartwell and Kastan, 1994, Science 266:1821-1828; Hunter, 1997, Cell 88:333-346; Sherr, 2000, Cancer Res. 60:3689-3695; Hanahan and Weinberg, 2000, Cell 100:57-70).

The cellular machinery and enzymatic components thereof involved in regulating and expressing the cell cycle are becoming known. One such component, the Cyclin D-dependent kinases, plays an important role in integrating extracellular signals into the cell cycle machinery (Sherr, 2000, Cancer Res. 60:3689-3695). D-type cyclins bind to and activate Cdk4 and Cdk6 during G1 (Matsushime et al., 1992, Cell 71:323-334; Meyerson and Harlow, 1994, Mol. Cell Biol. 14:2077-2086). This activation is followed by activation of Cdk2 in complex with cyclin E in late G1, which is essential for initiation of the S phase. Cdk2 also binds to cyclin A during S phase, playing a critical role in DNA replication.

The activities of Cdk4 and Cdk6 are regulated specifically by the Ink4-type inhibitors (p16^{Ink4a}, p15^{Ink4b}, p18^{Ink4c} and p19^{Ink4d}), while Cdk2 is inhibited by the Kip/Cip-type inhibitors (p21^{Cip1/Waf1}, p27^{KiP1} and p57^{Kip2}) (Sherr and Roberts, 1999, Genes Dev. 13:1501-1512; Kiyokawa and Koff, 1998, Curr. Top. Microbiol. Immunol. 227:105-120). Cyclin D/Cdk4 (Cdk6) phosphorylates retinoblastoma protein (Rb) and other Rb-related pocket binding proteins, including p107 and p130 (Ewen et al., 1993, Cell 73:487-497; Kato et al., 1993, Genes Dev. 7:331-342; Leng et al., 2002, Mol. Cell Biol. 22:2242-2254). Cdk4-dependent phosphorylation of specific sites of Rb is thought to facilitate Cdk2-dependent phosphorylation of other sites (Kitagawa et al., 1996, EMBO J. 15:7060-7069; Zarkowska and Mittnacht, 1997, J. Biol. Chem. 272:12738-12746; Connell-Crowley et al., 1997, Cell 8:287-301; Boylan et al., 1999, Exp. Cell Res. 248:110-114).

Hyperphosphorylation of Rb by Cdk molecules promotes conversion of the E2F transcription factors from repressor to transactivator status, which results in expression of a number of genes essential for S phase, including cyclins E and A (Nevins, 2001, Hum. Mol. Genet. 10:699-703). Furthermore, cyclin D/Cdk4 in proliferating cells binds to p21^{Cip1/Waf1} and p27^{KiP1} without being inactivated (Soos et al., 1996, Cell Growth Differ. 7:135-146; Blain et al., 1997, J. Biol. Chem. 272:25863-25872; Sherr and Roberts, 1999, Genes Dev. 13:1501-1512). Instead, these Kip/Cip proteins promote assembly of cyclin D/Cdk4 (LaBaer et al., 1997, Genes Dev. 11:847-862), suggesting the physical interaction with cyclin D/Cdk4 titrates p21 and p27 populations available for Cdk2 inhibition. Therefore, Cdk4 plays both catalytic and non-catalytic roles in controlling G1 progression.

A large number of human cancers show genetic alterations that deregulate cyclin D/Cdk4 (Hirama and Koeffler, 1995, Blood 86:841-854; Pestell et al., 1999, Endocr. Rev. 20:501-534; Sherr, 2000, Cancer Res. 60:3689-3695). For example, many glioblastomas, gliomas and sarcomas overexpress Cdk4 due to Cdk4 gene amplification (Khatib et al., 1993, Cancer Res. 53:5535-5541). Moreover, families genetically susceptible to melanoma have been found to carry germline mutations of Cdk4 at the Arg24 residue that render the kinase refractory to Ink4-dependent inhibition (Wolfel et al., 1995, Science 269:1281-1284; Zuo et al., 1996, Nat. Genet. 12:97-99). Tumor cells from various cancer types overexpress D-type cyclins. More frequent cancer-associated alterations are deletions, mutations and methylation of the Ink4a/Arf locus (Kamb et al., 1994, Science 264:436-440; Sherr, 1998, Genes Dev. 12:2984-2991; Sharpless and DePinho, 1999, Curr. Opin. Genet. Dev. 9:22-30).

The Ink4a/Arf locus contains two independent genes encoding p16Ink4a and p14^{Arf} (p19^{Arf} in mice), which share exons 2 and 3 on alternative reading frames (Quelle et al., 1995, Cell 83:993-1000). While p16^{Ink4a} inhibits Cdk4 and Cdk6, Arf protein interferes with Mdm2-dependent degradation of the tumor suppressor p53, leading to p53 stabilization (Pomerantz et al., 1998, Cell 92:713-723; Zhang et al., 1998, Cell 92:725-734; Stott et al., 1998, EMBO J. 17:5001-5014). Thus, inactivation of the Ink4a/Arf locus results in inappropriate activation of Cdk4 and rapid degradation of p53, both of which could contribute to tumorigenesis in distinct but cooperating manners. Consistent with this notion, mice deficient in both p16^{Ink4a} and p19^{Arf} (Serrano et al., 1996, Cell 85:277-37) or mice deficient in p19^{Arf} with intact p16^{Ink4a} (Kamijo et al., 1997, Cell 91:649-659) develop spontaneous tumors, while mice lacking p16^{Ink4a} with intact p19^{Arf} are susceptible to tumorigenesis to a lesser extent (Sharpless et al., 2001, Nature 413:86-91; Krimpenfort et al., 2001, Cell 413:83-86). These data suggest that activation of Cdk4 plays a critical role in tumorigenesis, and emphasize the need for Cdk4 inhibitors as anti-cancer agents.

### SUMMARY OF THE INVENTION

This invention provides methods of inhibiting tumor growth. Specifically, the invention provides such methods that inhibit tumor cell growth by inhibiting expression and/or activity of Cdk4 in tumor cells. In certain embodiments, Cdk4 expression and/or activity is inhibited in tumor cells by contacting the cells with a Cdk4 inhibitor. In one aspect, the tumor comprises cells that are completely deficient in p53 (p53−/−). In other aspects, the tumor comprises cells that express at least one copy of a mutated p53 gene or protein. In other aspects, the tumor cells express at least one copy of a mutated protein that participates in the p53 pathway. In a particular aspect, the Cdk4 inhibitor is an siRNA, a non-peptide molecule, or a protein that specifically inhibits the expression of a Cdk4 gene.

The invention also provides methods of treating an animal that has cancer, or bears growing tumor cells. In certain aspects, the animal is a human. Certain of the methods provided in this aspect of the invention comprise the step of administering a pharmaceutical composition to the animal, preferably a human patient, wherein the pharmaceutical composition comprises at least one inhibitor of Cdk4 expression or activity. In certain aspects, the pharmaceutical composition comprises a Cdk4 siRNA, a non-peptide molecule, or a peptide. In certain aspects, the animal, such as a human cancer patient, has a cancer that comprises (1) tumor cells that are completely p53 deficient (p53−/−); (2) tumor cells that comprise at least one mutated p53 gene or protein species; and/or (3) tumor cells that comprise at least one mutated gene or protein species that participates in the p53 pathway.

The invention further provides methods of protecting an animal, most preferably a human, from developing a disease or disorder comprising growing tumor cells such as cancer, comprising the step of administering to the animal a pharmaceutical composition comprising at least one inhibitor of Cdk4 expression or activity. In certain aspects, the pharmaceutical composition comprises a Cdk4 siRNA, a non-peptide molecule, or a peptide. In certain aspects, the animal has a tumor comprising (1) tumor cells that are completely p53 deficient (p53−/−); (2) tumor cells that comprise at least one mutated p53 gene or protein species; and/or (3) tumor cells that comprise at least one mutated gene or protein species that participates in the p53 pathway. In still other aspects, the animal is a human who has an increased risk for developing a cancer, for example, as a result of genetic predisposition, family history or environmental injury or insult.

In addition, the invention provides methods of screening for compounds that can inhibit tumor cell growth, wherein the tumor cell is completely p53 deficient (p53−/−) or comprises at least one mutated p53 gene or protein, the method comprising the steps of: (a) assaying Cdk4−/− cells for senescence in the presence of a test compound; (b) assaying Cdk4+/+ cells for senescence in the presence of the test compound; and (c) selecting the test compound as a tumor cell growth inhibitor if the Cdk4+/+ cells exhibit increased senescence in the presence of the compound, while Cdk4−/− cells show no increased senescence in the presence of the compound. In certain aspects, the method further comprises the step of assaying tumor cell growth in the presence and absence of the compound, and detecting decreased growth of tumor cells in the presence of the inhibitor compound.

The invention further provides pharmaceutical compositions comprising a tumor cell growth inhibitor compound identified according to a method of the invention. The invention also provides methods for treating an animal with cancer or having growing tumor cells, preferably a human cancer patient, the method comprising the step of administering a pharmaceutical composition of the invention to the animal, preferably a human cancer patient. In certain aspects, the animal is a cancer patient having a cancer that comprises (1) tumor cells that are completely p53 deficient (p53−/−); (2) tumor cells that comprise at least one mutated p53 gene or protein species; and/or (3) tumor cells that comprise at least one mutated gene or protein species that participates in the p53 pathway.

Furthermore, the invention provides methods of protecting an animal, preferably a human, from developing cancer, the method comprising the step of administering a pharmaceutical composition of the invention to the animal, preferably a human cancer patient to promote remission or prevent relapse, or a human without cancer having a risk of developing a disease or disorder characterized by growing tumor cells, such as cancer. In other aspects, the animal is a cancer patient having a tumor that comprises (1) tumor cells that are completely p53 deficient (p53−/−); (2) tumor cells that comprise at least one mutated p53 gene or protein species; and/or (3) tumor cells that comprise at least one mutated gene or protein species that participates in the p53 pathway. In still other aspects, the animal is a human who has an increased risk for developing a cancer, for example, as a result of genetic predisposition, family history or environmental injury or insult.

Specific preferred embodiments of the invention will become evident from the following more detailed description of certain preferred embodiments and the claims.

### DETAILED DESCRIPTION OF THE DRAWINGS

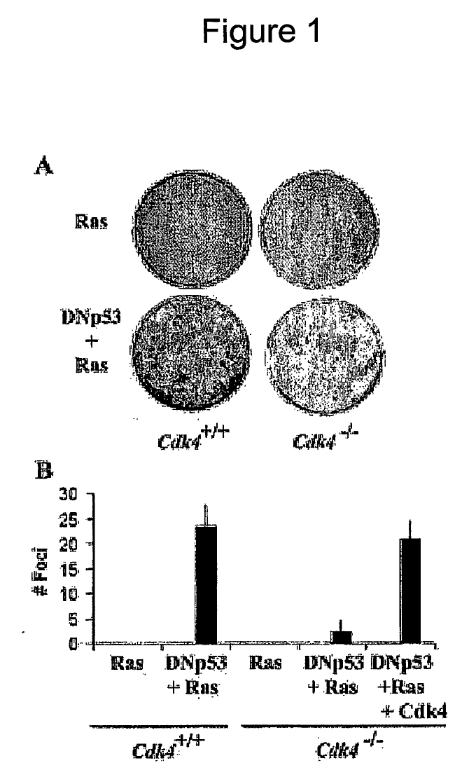

FIG. 1A is a photograph of tumor cell cultures showing foci formation in 60-mm dishes comprising passage 4 mouse embryonic fibroblasts (MEF) with the indicated genotypes infected with a retrovirus encoding H-Ras^{val12}, or with a virus encoding H-Ras^{val12} and a dominant-negative p53 (DNp53; amino acids 275-368) having an internal ribosomal entry site.

FIG. 1B is a graph showing the number of foci in the plates shown in FIG. 1A, expressed as the mean ±SEM from three independent MEF preparations.

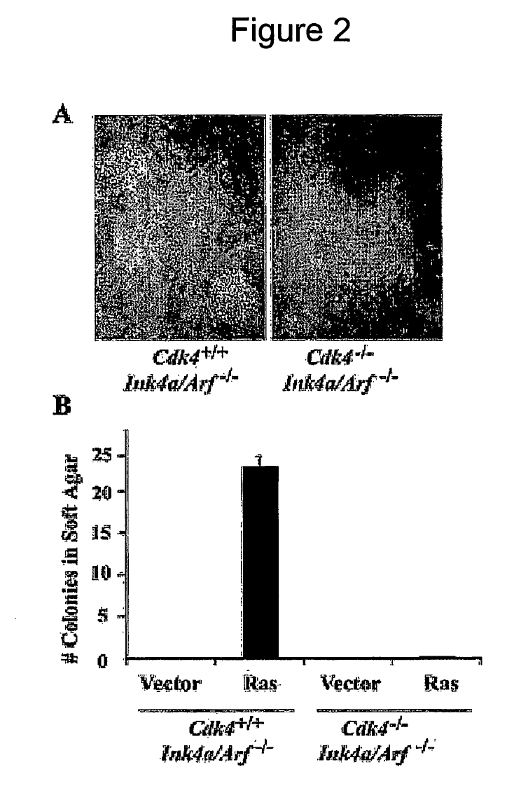

FIG. 2A is a photograph showing Cdk4^{+/+} and Cdk4^{-/-} cells plated in a medium containing soft agar and cultured for 21 days following retrovirus transduction of H-Ras^{val-12} and dominant-negative (DN) p53.

FIG. 2B is a graph showing the number of colonies per 10⁶ cells plated in the soft agar assays shown in FIG. 2A expressed as the mean ±SEM from three independent cell preparations.

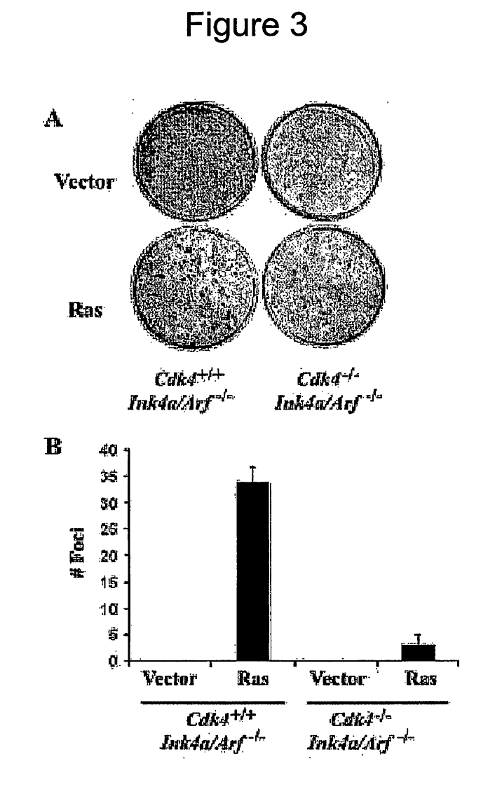

FIG. 3A is a photograph of tumor cell cultures showing foci formation in 60-mm dishes comprising passage 4 Cdk4−/− Ink4a/Arf−/− mouse embryonic fibroblasts (MEF) infected with a retrovirus encoding HRas^{val12}, or with a control virus with the pBabe-hygro vector.

FIG. 3B is a graph showing the number of foci in the plates shown in FIG. 3A, expressed as the mean ±SEM from three independent MEF preparations.

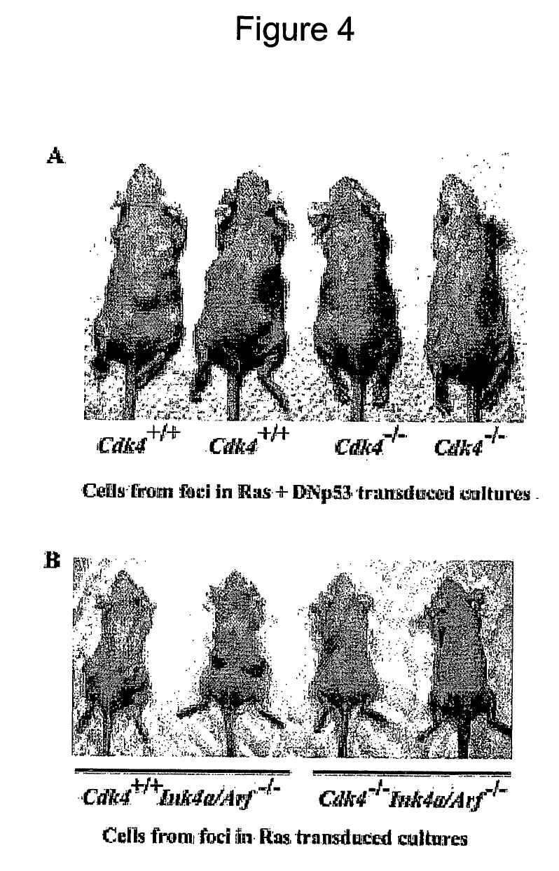

FIG. 4A is a photograph showing athymic mice injected with foci isolated from confluent cultures of Cdk4-null embryonic fibroblasts at 21 days following retrovirus transduction of H-Ras^{val12} and DNp53.

FIG. 4B is a photograph showing athymic mice injected with foci isolated from confluent cultures of Cdk4-null embryonic fibroblasts at 17 days following retrovirus transduction of H-Ras^{val-12}.

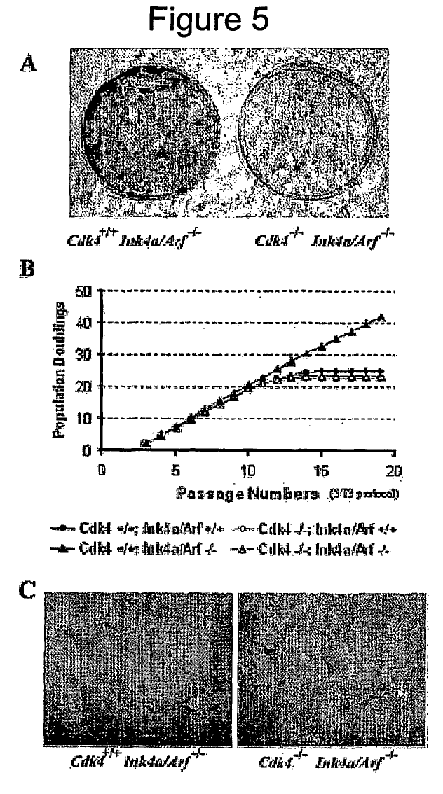

FIG. 5A is a photograph of tumor cell cultures showing colony growth of Cdk4^{+/+} Ink4a/Arf^{-/-} and Cdk4^{-/-} Ink4a/Arf^{-/-} mouse embryonic fibroblasts (MEF) at passage 11 plated at a low density (1×10³ cells per 60-mm dish), and cultured for 10 days. Colonies grown from isolated cells were stained with crystal violet.

FIG. 5B is a graph showing accumulated numbers of population doublings from three independent MEF preparations for each genotype, propagated in culture according to the 3T3 protocol.

FIG. 5C is a photograph of tumor cell cultures showing Cdk4^{+/+} Ink4a/Arf^{-/-} and Cdk4^{-/-} Ink4a/Arf^{-/-} MEF from passage 12, inoculated at 3×10³ cells per 60-mm dish and stained for senescence-associated β-galactosidase (SAβ-gal) after 10 days of growth.

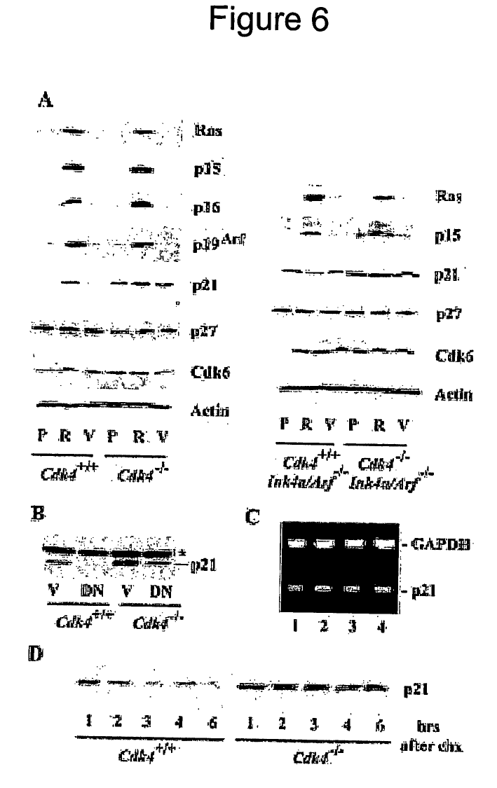

FIG. 6A is a photograph of autoradiograms showing Western blot analysis of protein extracts from Cdk4-null and Cdk4^{+/+} MEF infected with retrovirus constructed from pBabe-HRas^{val12} or pBabe-hygro control vector, selected for 72 hrs in the presence of 50 μg/ml hygromycin. P, uninfected proliferating cells (no selection); R, cells infected with H-Ras^{val12} retrovirus; V, cells infected with vector control virus.

FIG. 6B is a photograph of autoradiograms showing Western blot analysis of cells infected with retrovirus constructed from LXSN-dominant negative (DN) p53 or LXSN control vector (V). Infected cells were selected for 72 hrs in the presence of 2 μg/ml puromycin, and then analyzed by immunoblotting for the expression of p21^{Cip1/Waf1}. The asterisk indicates a band with nonspecific immunoreactivity.

FIG. 6C is a photograph of an ethidium bromide-stained electrophoretic gel showing expression of p21^{Cip1/Waf1} and GAPDH mRNA in exponentially proliferating cells at passage 4 as analyzed by RT-PCR. The genotypes of cells are: lane 1, Cdk4^{+/+} (wild-type); lane 2, Cdk4^{-/-}; lane 3, Cdk4^{+/+} Ink4a/Arf^{-/-}; lane 4, Cdk4^{-/-} Ink4a/Arf^{-/-}.

FIG. 6D is a photograph of autoradiograms showing Western blot analysis demonstrating that p21^{Cip1/Waf1} is stabilized in Cdk4^{-/-} cells. These data represent experiments using three independent cell preparations at passage 3 or 4 for each genotype.

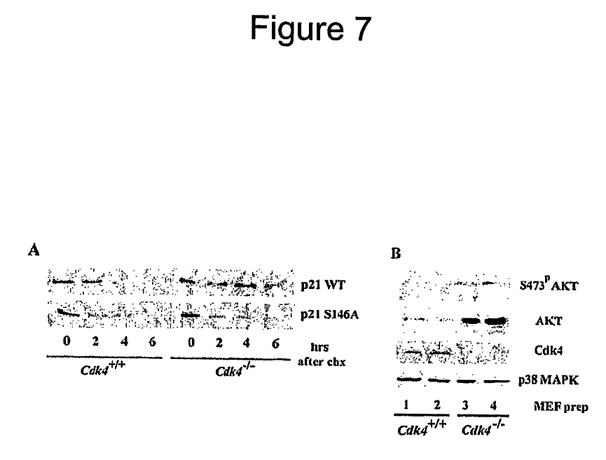

FIG. 7A is a photograph of autoradiograms showing Western blot analysis of wild type p21 and S146A mutant of human p21 in Cdk4^{-/-} and Cdk4^{+/+} MEFs after retroviral transduction of exogenous p21 or S146A mutant p21 and treatment with cycloheximide (chx).

FIG. 7B is a photograph of autoradiograms showing Western blot analysis of a number of proteins in Cdk4^{-/-} and Cdk4^{+/+} MEFs.

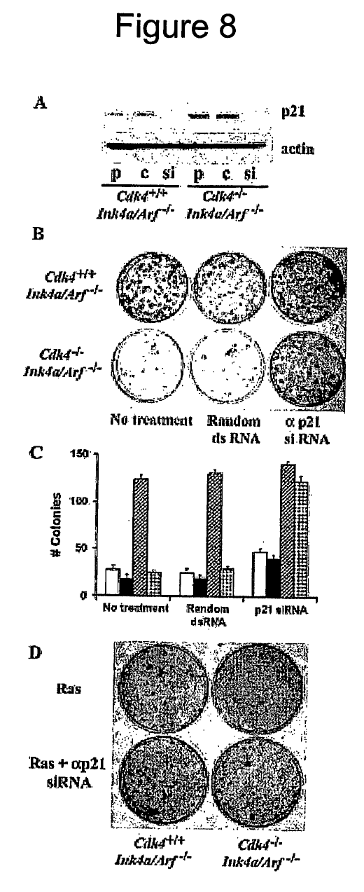

FIG. 8A is a photograph of autoradiograms showing Western blot analysis with anti-p21^{Cip1/Waf1} and anti-actin antibodies performed on protein extracts from Cdk4^{+/+} Ink4a/Arf^{-/-}; and Cdk4^{-/-} Ink4a/Arf^{-/-} MEF transfected with small interfering RNA (siRNA) that specifically targets p21^{Cip1/Waf1} mRNA or with random double stranded (ds) RNA. p, non-transfected proliferating cells; c, cells transfected with control random dsRNA; si, anti-p21^{Cip1/Waf1} siRNA.

FIG. 8B is a photograph of tumor cell cultures showing cells at passage 10 plated at a density of 1×10³ cells/plate 24 hr after being transfected with the anti-p21 siRNA or control dsRNA.

FIG. 8C is a graph representing the number of colonies (>2 mm) counted at 10 days post-plating expressed as the mean ±SEM from three independent cell preparations. Open columns, Cdk4^{+/+}; closed columns, Cdk4^{-/-}; hatched columns, Cdk4^{+/+} Ink4a/Arf^{-/-}; dotted columns, Cdk4^{+/+} Ink4a/Arf^{-/-}.

FIG. 8D is a photograph of tumor cell cultures showing cells at passage 4 transfected with anti-p21^{Cip1/Waf1} siRNA or control dsRNA, and 24 hr later infected with H-Ras^{val-12} retrovirus. Foci formation was scored at 15 days post transfection.

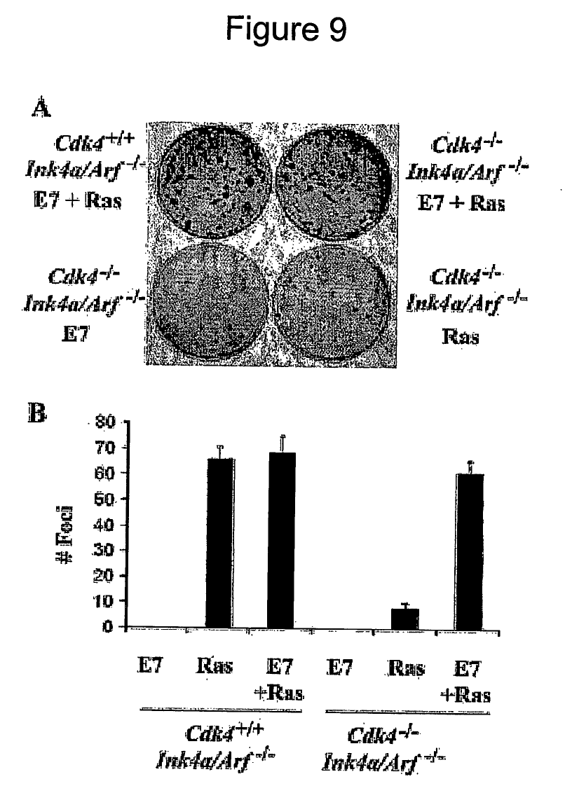

FIG. 9A is a photograph of tumor cell cultures showing passage 4 mouse embryonic fibroblasts (MEF) with indicated genotypes after infection with E7 retrovirus or control virus, followed by infection with H-Ras^{val-12} retrovirus or control virus with a 24-hr interval. Cells were then cultured in the medium containing 5% FBS for 17 days.

FIG. 9B is a graph showing the numbers of foci per 60-mm dish in the assays expressed as the mean ±SEM from three independent MEF preparations.

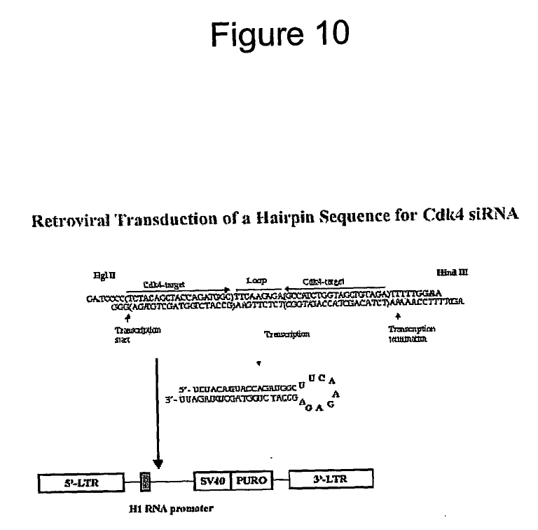

FIG. 10 is a schematic diagram showing retroviral transduction of a hairpin sequence for Cdk4 siRNA. The Cdk4 target sequence corresponds to nucleotides 492-509 of human cdk4 cDNA, which also is identical to the same region of the mouse cDNA.

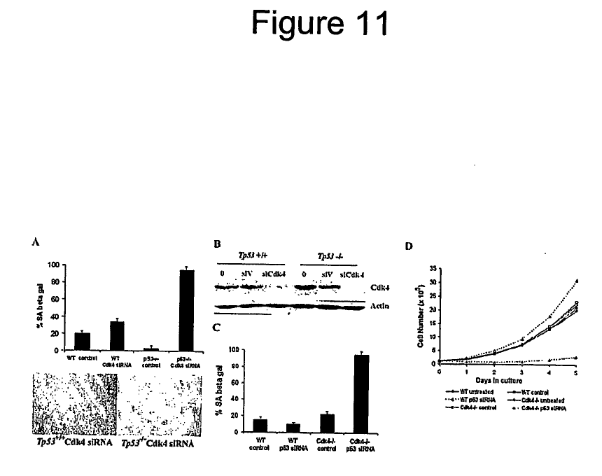

FIG. 11 is a graph showing retroviral transduction of anti-Cdk4 or p53 siRNA in various MEFs; a photomicrograph of the cells from which the graph was constructed is also shown. MEFs at passage 3 were infected with the indicated retroviruses, followed by 72-h selection with puromycin (2 μg/ml) that started at 24 h post-infection. Senescence (shown in FIGS. 11A and 11C) was evaluated by SA-β-gal staining and effects of Cdk4 siRNA were examined by immunoblotting FIG. 11B). For cell growth curves (shown in FIG. 11D), cells were infected with concentrated viruses, replated at 72 h postinfection, and cell counts were determined daily.

### DETAILED DESCRIPTION OF THE PREFERRED EMBODIMENTS

This invention provides methods for inhibiting growth of tumor cells. In certain and preferred embodiments, the methods of the invention comprise the step of contacting the tumor cell with at least one inhibitor of Cdk4 expression or activity. Preferably, the tumor cell is completely deficient in p53 (p53^{-/-}), comprises at least one copy of a mutated p53 gene, comprises a mutated p53 protein, or comprises a mutated gene or protein that participates in the p53 cellular pathway. The term “p53 pathway” is intended to encompass genes and proteins involved in or that interact with p53 in a cell to regulate cell growth, as understood in the art (see, for example, Drayton & Peters, 2002, Curr Opin Genet Dev. 12:98-104; Sharpless & DePinho, 2002. Cell. 110:9-12; Lowe & Sherr, 2003, Curr Opin Genet Dev. 13:77-83; and Oren, 2003, Cell Death Differ. 10:431-42. As provided by the invention, said methods can be used to inhibit tumor cells in vitro or in vivo (e.g. a cell that has not been removed from a patient).

As used herein, an “inhibitor” can be any chemical compound, including but not limited to a nucleic acid molecule, or a peptide or polypeptide such as an antibody having immunological specificity against a gene product, that can reduce activity of a gene product or interfere with expression (including transcription, processing, translation, and post-translational modification) of a gene. An inhibitor as provided by the invention, for example, can inhibit directly or indirectly the activity of a protein that is encoded by a gene (i.e., a gene product). Direct inhibition can be accomplished, for example, by binding to a protein and thereby preventing the protein from binding an intended target, such as a receptor, or by inhibiting an enzymatic or other activity of the protein, either competitively, non-competitively or uncompetitively. Indirect inhibition can be accomplished, for example, by binding to a protein's intended target, such as a receptor or binding partner, thereby blocking or reducing activity of the protein. Furthermore, an inhibitor according to the invention can inhibit a gene by reducing or inhibiting expression of the gene, inter alia, by interfering with mRNA encoded by the gene thereby blocking translation of the gene product.

In certain embodiments of the invention, a Cdk4 activity inhibitor can be, for example, a small molecule, a protein such as an antibody or immunologically-reactive fragment thereof, or a nucleic acid including an antisense oligonucleotide, an siRNA molecule, or an shRNA molecule. Such inhibitors may be known in the art or as described herein. In addition, such inhibitors can be specifically designed using the methods described herein or using methods known in the art. For example, antibodies to proteins encoded by a gene shown in Table 1 can be generated by conventional means as described, for example, in “Antibodies: A Laboratory Manual” by Harlow and Lane (Cold Spring Harbor Press, 1988), which is hereby incorporated by reference. Non-limiting examples of small molecule Cdk inhibitors include but are not limited to olomoucine, butyrolactone, certain flavonoids, staurosporine and its related compound UCN-01, suramin, toyocamycin, certain ellipticines, certain paullones and certain pyridopyrimidines (as disclosed, inter alia, in Ortega et al., 2002, Biochim Biophys Acta. 1602: 73-87; Walker. 1998, Curr Top Microbiol Immunol. 227: 149-165; and Garrett & Fattaey. 1999, Curr Opin Genet Dev. 9: 104-111). All these compounds have broad spectra against multiple Cdk proteins and other protein kinases. Compounds that are relatively more specific inhibitors of Cdk4 include a triaminopyrimidine derivative CINK4 (Soni et al., 2001, J Natl Cancer Inst. 93: 436-446), PD0183812 (Fry et al., 2001, J Biol Chem. 276: 16617-16623) and AG12275 (Tetsu & McCormick, 2003, Cancer Cell. 3: 233-245; and Toogood, 2001, Med Res Rev. 6: 487-498). However, none of these compounds have been examined for specific growth inhibition in p53-deficient tumor cells or in vivo tumors lacking p53 function.

Also provided are related compounds within the understanding of those with skill in the art, such as chemical mimetics, organomimetics or peptidomimetics. As used herein, the terms “mimetic,” “peptide mimetic,” “peptidomimetic,” “organomimetic” and “chemical mimetic” are intended to encompass peptide derivatives or analogues and chemical compounds having an arrangement of atoms is a three-dimensional orientation that is equivalent to that of a Cdk4 inhibitor of the invention. It will be understood that the phrase “equivalent to” as used herein is intended to encompass compounds having substitution of certain atoms or chemical moieties in said Cdk4 inhibitor with moieties having bond lengths, bond angles and arrangements thereof in the mimetic compound that produce the same or sufficiently similar arrangement or orientation of said atoms and moieties to have the biological function of the Cdk4 inhibitors of the invention resulting in such peptido-, organo- and chemical mimetics of the peptides of the invention having substantial biological activity. In the peptide mimetics of the invention, the three-dimensional arrangement of the chemical constituents is structurally and/or functionally equivalent to the three-dimensional arrangement of the Cdk4 inhibitor. These terms are used according to the understanding in the art, as illustrated for example by Fauchere, 1986, Adv. Drug Res. 15: 29; Veber & Freidinger, 1985, TINS p. 392; and Evans et al., 1987, J. Med. Chem. 30: 1229, incorporated herein by reference.

It is understood that a pharmacophore exists for the biological activity of each Cdk4 inhibitor of the invention. A pharmacophore is understood in the art as comprising an idealized, three-dimensional definition of the structural requirements for biological activity. Peptido-, organo- and chemical mimetics can be designed to fit each pharmacophore with current computer modeling software (computer aided drug design). Said mimetics are produced by structure-function analysis, based on the positional information from the substituent atoms in the Cdk4 inhibitors of the invention.

Mimetic analogs of the Cdk4 inhibitors of the invention may be obtained using the principles of conventional or rational drug design (see, Andrews et al., 1990, Proc. Alfred Benzon Symp. 28: 145-165; McPherson, 1990, Eur. J. Biochem. 189:1-24; Hol et al., 1989a, in MOLECULAR RECOGNITION: CHEMICAL AND BIOCHEMICAL PROBLEMS, (Roberts, ed.); Royal Society of Chemistry; pp. 84-93; Hol, 1989b, Arzneim-Forsch. 39:1016-1018; Hol, 1986, Agnew Chem. Int. Ed. Engl. 25: 767-778, the disclosures of which are herein incorporated by reference). In accordance with the methods of conventional drug design, the desired mimetic molecules are obtained by randomly testing molecules whose structures have an attribute in common with the structure of one or a plurality of known Cdk4 inhibitors. The quantitative contribution that results from a change in a particular group of a binding molecule can be determined by measuring the biological activity of the putative mimetic in comparison with the Cdk4 inhibiting activity of the compound. In a preferred embodiment of rational drug design, the mimetic is designed to share an attribute of the most stable three-dimensional conformation of the Cdk4 inhibitor. Thus, for example, the mimetic may be designed to possess chemical groups that are oriented in a way sufficient to cause ionic, hydrophobic, or van der Waals interactions that are similar to those exhibited by the Cdk4-inhibiting compounds of the invention, as disclosed herein.

The preferred method for performing rational mimetic design employs a computer system capable of forming a representation of the three-dimensional structure of the Cdk4 inhibitor, such as those exemplified by Hol, 1989a, ibid.; Hol, 1989b, ibid.; and Hol, 1986, ibid. Molecular structures of the peptido-, organo- and chemical mimetics of the Cdk4 inhibitors of the invention are produced according to those with skill in the art using computer-assisted design programs commercially available in the art. Examples of such programs include SYBYL 6.5®, HQSAR™, and ALCHEMY 2000™ (Tripos); GALAXY™ and AM2000™ (AM Technologies, Inc., San Antonio, Tex.); CATALYST™ and CERIUS™ (Molecular Simulations, Inc., San Diego, Calif.); CACHE PRODUCTS™, TSAR™, AMBER™, and CHEM-X™ (Oxford Molecular Products, Oxford, Calif.) and CHEMBUILDER3D™ (Interactive Simulations, Inc., San Diego, Calif.).

The peptido-, organo- and chemical mimetics produced using the Cdk4 inhibitors disclosed herein using, for example, art-recognized molecular modeling programs are produced using conventional chemical synthetic techniques, most preferably designed to accommodate high throughput screening, including combinatorial chemistry methods. Combinatorial methods useful in the production of the peptido-, organo- and chemical mimetics of the invention include phage display arrays, solid-phase synthesis and combinatorial chemistry arrays, as provided, for example, by SIDDCO, Tuscon, Ariz.; Tripos, Inc.; Calbiochem/Novabiochem, San Diego, Calif.; Symyx Technologies, Inc., Santa Clara, Calif.; Medichem Research, Inc., Lemont, Ill.; Pharm-Eco Laboratories, Inc., Bethlehem, Pa.; or N.V. Organon, Oss, Netherlands. Combinatorial chemistry production of the peptido-, organo- and chemical mimetics of the invention are produced according to methods known in the art, including but not limited to techniques disclosed in Terrett, 1998, COMBINATORIAL CHEMISTRY, Oxford University Press, London; Gallop et al., 1994, “Applications of combinatorial technologies to drug discovery. 1. Background and peptide combinatorial libraries,” J. Med. Chem. 37: 1233-51; Gordon et al., 1994, “Applications of combinatorial technologies to drug discovery. 2. Combinatorial organic synthesis, library screening strategies, and future directions,” J. Med. Chem. 37: 1385-1401; Look et al., 1996, Bioorg. Med. Chem. Lett. 6: 707-12; Ruhland et al., 1996, J. Amer. Chem. Soc. 118: 253-4; Gordon et al., 1996, Acc. Chem. Res. 29: 144-54; Thompson & Ellman, 1996, Chem. Rev. 96: 555-600; Fruchtel & Jung, 1996, Angew. Chem. Int. Ed. Engl. 35: 1742; Pavia, 1995, “The Chemical Generation of Molecular Diversity”, Network Science Center, www.netsci.org; Adnan et al., 1995, “Solid Support Combinatorial Chemistry in Lead Discovery and SAR Optimization,” Id., Davies and Briant, 1995, “Combinatorial Chemistry Library Design using Pharmacophore Diversity,” Id., Pavia, 1996, “Chemically Generated Screening Libraries: Present and Future,” Id.; and U.S. Pat. Nos. 5,880,972 to Horlbeck; 5,463,564 to Agrafiotis et al.; 5,331,573 to Balaji et al.; and 5,573,905 to Lerner et al.

In a preferred embodiment, Cdk4 inhibitors as provided by the invention are species of short interfering RNA (siRNA). The term “short interfering RNA” or “siRNA” as used herein refers to a double stranded nucleic acid molecule capable of RNA interference or “RNAi”, as disclosed, for example, in Bass, 2001, Nature 411: 428-429; Elbashir et al., 2001, Nature 411: 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914. As used herein, siRNA molecules need not be limited to those molecules containing only RNA, but further encompasses chemically modified nucleotides and non-nucleotides having RNAi capacity or activity.

RNA interference refers to the process of sequence-specific post-transcriptional gene silencing in animals mediated by short interfering RNAs (siRNA) (Fire et al., 1998, Nature 391:806). The presence of long dsRNAs in cells stimulates the activity of a ribonuclease III enzyme referred to as “dicer.” Dicer is involved in processing of the dsRNA into short pieces of dsRNA known as short interfering RNAs (siRNA) (Berstein et al., 2001, Nature 409:363). Short interfering RNAs derived from dicer activity are typically about 21-23 nucleotides in length and comprise about 19 base pair duplexes. Dicer has also been implicated in the excision of 21 and 22 nucleotide small temporal RNAs (stRNA) from precursor RNA of conserved structure that are implicated in translational control (Hutvagner et al., 2001, Science 293:834). The RNAi response also features an endonuclease complex containing an siRNA, commonly referred to as an RNA-induced silencing complex (RISC), which mediates cleavage of single-stranded RNA having sequence homologous to the siRNA. Cleavage of the target RNA takes place in the middle of the region complementary to the guide sequence of the siRNA duplex (Elbashir et al., 2001, Genes Dev. 15:188).

Short interfering RNA mediated RNAi has been studied in a variety of systems. Fire et al. were the first to observe RNAi in C. elegans (1998, Nature 391:806). Wianny and Goetz described RNAi mediated by dsRNA in mouse embryos (1999, Nature Cell Biol. 2:70). Hammond et al. described RNAi in Drosophila cells transfected with dsRNA (2000, Nature 404:293). Elbashir et al. describe RNAi induced by introduction of duplexes of synthetic 21-nucleotide RNAs in cultured mammalian cells including human embryonic kidney and HeLa cells (2001, Nature 411:494). Recent work in Drosophila embryo lysates has revealed certain requirements for siRNA length, structure, chemical composition, and sequence that are essential to mediate efficient RNAi activity.

These studies have shown that siRNA duplexes comprising 21 nucleotides are most active when containing two nucleotide 3′-overhangs. Furthermore, substitution of one or both siRNA strands with 2′-deoxy or 2′-O-methyl nucleotides abolishes RNAi activity, whereas substitution of 3′-terminal siRNA nucleotides with deoxy nucleotides was shown to be tolerated. Mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end (Elbashir et al., 2001, EMBO J. 20:6877). Other studies have indicated that a 5′-phosphate on the target-complementary strand of a siRNA duplex is required for siRNA activity and that ATP is utilized in cells to maintain the 5′-phosphate moiety on the siRNA (Nykanen et al., 2001, Cell 107:309). However siRNA molecules lacking a 5′-phosphate are active when introduced exogenously, suggesting that 5′-phosphorylation of siRNA constructs may occur in vivo.

Cdk inhibition accomplished by RNAi-based knockdown of Cdk4 expression has advantages over pharmacological Cdk inhibitors. These include: (1) high specificity, because pharmacological inhibitors tend to inhibit broad spectrum of related kinases, e.g. Cdk 1 and Cdk2, which could cause side effects by inhibiting normal cell proliferation and function; (2) low toxicity, as evidenced by normal development observed in Cdk4 knockout mice and normal proliferation rates observed in Cdk4-null cells; moreover, retroviral transduction can be used to target anti-Cdk4 RNAi in precancerous or cancerous lesions in vivo, after appropriate modifications; (3) long-term effect, because long-term gene silencing can be expected since retroviral transduction causes chromosomal integration of the mini-gene that express a loop structure of anti-Cdk4 RNA. In contrast, pharmacological inhibitors should be administered continuously in order to obtain long-term inhibition of Cdk4. RNAi-based Cdk4 knockdown can also be used for chemoprevention of cancer-prone patients, e.g. to reduce a risk of breast cancer in Brcal-mutant humans.

In preferred embodiments, a Cdk4 siRNA is designed and constructed as described in Example 8 herein, which describes production of a 21-basepair siRNA that corresponds to nucleotide residues 489-509 of the mouse Cdk4 coding sequence. The Cdk4 siRNA described in Example 8 is an exemplary Cdk4 siRNA having a nucleotide sequence as shown in SEQ ID NO: 6, and was constructed using the methods described in Elbashir et al. (2001, Genes Dev. 15:188-200; 2001, Nature 411:494-498), which is incorporated herein by reference.

In certain embodiments, the invention provides expression vectors comprising a nucleic acid sequence encoding at least one siRNA molecule of the invention, in a manner that allows expression of the siRNA molecule. For example, the vector can contain sequence(s) encoding both strands of an siRNA molecule comprising a duplex. The vector can also contain sequence(s) encoding a single nucleic acid molecule that is self-complementary and thus forms an siRNA molecule. Non-limiting examples of such expression vectors are described in Paul et al., 2002, Nature Biotechnology 19:505; Miyagishi and Taira, 2002, Nature Biotechnology 19:497; Lee et al., 2002, Nature Biotechnology 19:500; and Novina et al., 2002, Nature Medicine, online publication June 3.

In other embodiments, the invention provides mammalian cells, for example, human cells, comprising an expression vector of the invention. In further embodiments, the expression vector comprising said cells of the invention comprises a sequence for an siRNA molecule having complementarity to at least a portion of human or mouse Cdk4 coding sequence, wherein expression of said siRNA in the cell inhibits Cdk4 expression therein. In other embodiments, expression vectors of the invention comprise a nucleic acid sequence encoding two or more siRNA molecules, which can be the same or different. In other embodiments of the invention, siRNA molecules, preferably Cdk4-specific siRNA molecules, are expressed from transcription units inserted into DNA or RNA vectors.

The invention provides methods of screening for compounds that inhibit tumor cell growth, wherein the tumor cell completely p53 deficient (p53−/−); comprises at least one mutated p53 gene or protein species; and/or comprises at least one mutated gene or protein species that participates in the p53 pathway, the method comprising the steps of: (a) assaying Cdk4^{-/-} cells for senescence in the presence of a test compound; (b) assaying Cdk4^{+/+} cells for senescence in the presence of the test compound; and (c) selecting the test compound as a tumor cell growth inhibitor if the Cdk4^{+/+} cells exhibit increased senescence in the presence of the compound, while Cdk4^{-/-} cells show no increased senescence in the presence of the compound. In certain aspects, the method further comprises the step of assaying tumor cell growth in the presence and absence of the compound, and detecting decreased growth of tumor cells in the presence of the inhibitor compound.

Cdk4^{-/-} and Cdk4^{+/+} cells are described, for example, in Example 1 below. Senescence assays are performed, for example, as described in the Examples below. Tumor cells that are p53^{-/-} are known in the art and include, for example, those cells shown and described in Table 1. The Saos-2 cells, HCT116 cells, MDA-MB-468 cells, MDA-MB-231 cells, T47D cells and OVCAR-3 cells are available from the American Type Culture Collection, Manassas, Va. The OVCAR-5 cells are available from Dr. T. Hamilton (Fox Chase Cancer Institute, Philadelphia, Pa.).

TABLE 1

| Cell Line | Origin | p53 status |
|---|---|---|
| Saos-2 | Osteosarcoma | Deleted (−/−) |
| HCT116 | Colon cancer | Deleted (−/−) |
| MDA-MB-468 | Breast cancer | Mutated |
| MDA-MB-231 | Breast cancer | Mutated |
| T-47D | Breast cancer | Mutated |
| OVCAR-3 | Ovarian cancer | Mutated |
| OVCAR-5 | Ovarian cancer | Mutated |

Cell growth can be assayed as described herein or using any conventional cell growth assay known in the art.

In certain embodiments, siRNA molecules according to the invention can comprise a delivery vehicle, including inter alia liposomes, for administration to a subject, carriers and diluents and their salts, and can be present in pharmaceutical compositions. Methods for the delivery of nucleic acid molecules are described, for example, in Akhtar et al., 1992, Trends Cell Bio. 2:139; Delivery Strategies for Antisense Oligonucleotide Therapeutics, ed. Akhtar, 1995, Maurer et al., 1999, Mol. Membr. Biol. 16:129-140; Hofland and Huang, 1999, Handb. Exp. Pharmacol., 137:165-192; and Lee et al., 2000, ACS Symp. Ser. 752:184-192, all of which are incorporated herein by reference. Beigelman et al., U.S. Pat. No. 6,395,713 and Sullivan et al., PCT WO 94/02595, further describe general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other delivery vehicles, such as hydrogels, cyclodextrins, biodegradable nanocapsules, and bioadhesive microspheres, or by proteinaceous vectors (see, for example, O'Hare and Normand, International PCT Publication No. WO 00/53722).

Alternatively, the nucleic acid/vehicle combination can be locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., 1999, Clin. Cancer Res. 5:2330-2337 and Barry et al., International PCT Publication No. WO 99/31262. Many examples in the art describe delivery methods of oligonucleotides by osmotic pump, (see Chun et al., 1998, Neuroscience Letters 257:135-138, D'Aldin et al., 1998, Mol. Brain Research 55:151-164, Dryden et al., 1998, J. Endocrinol. 157:169-175, Ghirnikar et al., 1998, Neuroscience Letters 247:21-24) or direct infusion (Broaddus et al., 1997, Neurosurg. Focus 3, article 4). Other delivery routes include, but are not limited to oral delivery (such as in tablet or pill form) and/or intrathecal delivery (Gold, 1997, Neuroscience 76:1153-1158). More detailed descriptions of nucleic acid delivery and administration are provided in Sullivan et al., PCT WO 94/02595, Draper et al., PCT WO93/23569, Beigelman et al., PCT WO99/05094, and Klimuk et al., PCT WO99/04819, all of which are incorporated by reference herein.

In some embodiments, the invention provides pharmaceutical compositions comprising a Cdk4 inhibitor. In one embodiment, a pharmaceutical composition of the invention can comprise a Cdk4 inhibitor, either a Cdk4 inhibitor known in the art or a compound identified as a Cdk4 inhibitor using a screening method of the invention, in admixture with a pharmaceutically or physiologically acceptable formulation agent selected for suitability with the mode of administration. In other embodiments, a pharmaceutical composition of the invention can comprise a therapeutically effective amount of a nucleic acid molecule of the invention, such as a nucleic acid molecule that comprises SEQ ID NO: 6 or any Cdk4 siRNA that inhibits Cdk4 activity, in admixture with a pharmaceutically or physiologically acceptable formulation agent selected for suitability with the mode of administration.

The invention thus provides Cdk4 inhibitors, and methods for identifying said inhibitors, that are useful for inhibiting tumor cell growth. In certain embodiments, the methods of the invention for inhibiting tumor cell growth are carried out in combination with a chemotherapeutic agent or agents. Chemotherapeutic agents are known in the art, and include, for example, cis-platin, paclitaxel, carboplatin, etoposide, hexamethylamine, melphalan, and anthracyclines.

In other embodiments, the invention provides methods of treating an animal, most preferably a human patient, bearing a tumor or growing tumor cells by administering a pharmaceutical composition of the invention to the patient. In one embodiment, a “patient” can be an individual who has a cancer, wherein the cancer that comprises (1) tumor cells that are completely p53 deficient (p53−/−); (2) tumor cells that comprise at least one mutated p53 gene or protein; and/or (3) tumor cells that comprise at least one mutated gene or protein that participates in the p53 pathway. In another embodiment, a “patient” can be an individual who has an increased risk for developing cancer, for example, as a result of genetic predisposition, family history or environmental injury or insult. For example, the patient can have a mutated gene that is associated with an increased risk of developing a cancer, such as the Brcal gene, or other family history-related predisposition to developing cancer.

In a particular embodiment, invention provides methods of protecting a patient from developing cancer comprising the step of administering a pharmaceutical composition to the patient, wherein the pharmaceutical composition comprises at least one inhibitor of Cdk4 expression or activity. As used herein, “protecting” refers to decreasing the likelihood and/or risk that the patient treated with a pharmaceutical composition of the invention will develop a tumor.

Acceptable formulation materials for a pharmaceutical composition of the invention preferably are nontoxic to recipients at the dosages and concentrations employed.

The pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants. See Remington 's Pharmaceutical Sciences (18th Ed., A. R. Gennaro, ed., Mack Publishing Company 1990.

The optimal pharmaceutical composition can be determined by a skilled artisan depending upon, for example, the intended route of administration, delivery format, and desired dosage. See, e.g., Remington's Pharmaceutical Sciences, supra. Such compositions can influence the physical state, stability, rate of in vivo release, and rate of in vivo clearance of a Cdk4 inhibitor of the invention.

The primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute. In one embodiment of the invention, pharmaceutical compositions of the invention can be prepared for storage by mixing the selected composition having the desired degree of purity with optional formulation agents (Remington's Pharmaceutical Sciences, supra) in the form of a lyophilized cake or an aqueous solution. Further, the composition can be formulated as a lyophilizate using appropriate excipients such as sucrose.

The pharmaceutical compositions can be selected for parenteral delivery. Alternatively, the compositions can be selected for inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use in the invention can be in the form of a pyrogen-free, parenterally acceptable, aqueous solution comprising the desired molecule of the invention in a pharmaceutically acceptable vehicle. A particularly suitable vehicle for parenteral injection is sterile distilled water in which the molecule is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which may then be delivered via a depot injection. Hyaluronic acid can also be used, which can have the effect of promoting sustained duration in the circulation. Other suitable means for the introduction of the desired molecule include implantable drug delivery devices.

In one embodiment, a pharmaceutical composition can be formulated for inhalation. For example, a Cdk4 inhibitor of the invention can be formulated as a dry powder for inhalation. Inhalation solutions can also be formulated with a propellant for aerosol delivery. In yet another embodiment, solutions can be nebulized. Pulmonary administration is further described in PCT Pub. No. WO 94/20069, which describes the pulmonary delivery of chemically modified proteins.

In other embodiments, certain formulations can be administered orally. In one embodiment of the invention, Cdk4 inhibitors of the invention that are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the molecule or modulator of the invention. Diluents, flavorings, low melting point waxes, vegetable oils, lubricants, suspending agents, tablet disintegrating agents, and binders may also be employed.

Another pharmaceutical composition can involve an effective quantity of Cdk4 inhibitors of the invention in a mixture with non-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving Cdk4 inhibitors of the invention in sustained- or controlled-delivery formulations. Techniques for formulating a variety of other sustained- or controlled-delivery means, such as liposome carriers, bio-erodible microparticles or porous beads and depot injections, are also known to those skilled in the art. See, e.g., PCT/US93/00829, which describes the controlled release of porous polymeric microparticles for the delivery of pharmaceutical compositions.

Additional examples of sustained-release preparations include semipermeable polymer matrices in the form of shaped articles, e.g. films, or microcapsules. Sustained release matrices may include polyesters, hydrogels, polylactides (U.S. Pat. No. 3,773,919 and European Patent No. 058481), copolymers of L-glutamic acid and gamma ethyl-L-glutamate (Sidman et al., 1983, Biopolymers 22:547-56), poly(2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., supra) or poly-D(−)-3-hydroxybutyric acid (European Patent No. 133988). Sustained-release compositions may also include liposomes, which can be prepared by any of several methods known in the art. See, e.g., Eppstein et al., 1985, Proc. Natl. Acad. Sci. USA 82:3688-92; and European Patent Nos. 036676, 088046, and 143949.

A pharmaceutical composition to be used for in vivo administration typically must be sterile. This may be accomplished by filtration through sterile filtration membranes. Where the composition is lyophilized, sterilization using this method may be conducted either prior to, or following, lyophilization and reconstitution. The composition for parenteral administration can be stored in lyophilized form or in a solution. In addition, parenteral compositions generally are placed into a container having a sterile access port, for example, an intravenous solution bag or vial having a stopper pierceable by a hypodermic injection needle.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

In a specific embodiment, the invention is directed to kits for producing a single-dose administration unit. The kits can each contain both a first container having a dried protein and a second container having an aqueous formulation. Also included within the scope of this invention are kits containing single and multi-chambered pre-filled syringes (e.g., liquid syringes and lyosyringes).

The effective amount of a pharmaceutical composition of the invention to be employed therapeutically will depend, for example, upon the therapeutic context and objectives. One skilled in the art will appreciate that the appropriate dosage levels for treatment will thus vary depending, in part, upon the molecule delivered, the indication for which the composition is being used, the route of administration, and the size (body weight, body surface, or organ size) and condition (the age and general health) of the patient. Accordingly, the clinician may titer the dosage and modify the route of administration to obtain the optimal therapeutic effect. A typical dosage may range from about 0.1 μg/kg to up to about 100 mg/kg or more, depending on the factors mentioned above. In other embodiments, the dosage may range from 0.1 μg/kg up to about 100 mg/kg; or 1 μg/kg up to about 100 mg/kg; or 5 μg/kg up to about 100 mg/kg.

The frequency of dosing will depend upon the pharmacokinetic parameters of the Cdk4 inhibitors of the invention in the formulation being used. Typically, a clinician will administer the composition until a dosage is reached that achieves the desired effect. The composition may therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

The route of administration of the pharmaceutical composition is in accord with known methods, e.g., orally; through injection by intravenous, intraperitoneal, intracerebral (intraparenchymal), intracerebroventricular, intramuscular, intraocular, intraarterial, intraportal, or intralesional routes; by sustained release systems; or by implantation devices. Where desired, the compositions may be administered by bolus injection or continuously by infusion, or by implantation device.

Alternatively or additionally, the composition may be administered locally via implantation of a membrane, sponge, or other appropriate material onto which the desired molecule has been absorbed or encapsulated. Where an implantation device is used, the device may be implanted into any suitable tissue or organ, and delivery of the desired molecule may be via diffusion, timed-release bolus, or continuous administration.

In some cases, it may be desirable to use pharmaceutical compositions of the invention in an ex vivo manner. In such instances, cells, tissues, or organs that have been removed from the patient are exposed to the pharmaceutical compositions after which the cells, tissues, or organs are subsequently implanted back into the patient.

In other cases, Cdk4 inhibitors of the invention can be delivered by implanting certain cells that have been genetically engineered, using methods such as those described herein, to express and secrete the Cdk4 inhibitors of the invention. Such cells can be animal or human cells, and can be autologous, heterologous, or xenogeneic. Optionally, the cells can be immortalized. In order to decrease the chance of an immunological response, the cells can be encapsulated to avoid infiltration of surrounding tissues. The encapsulation materials are typically biocompatible, semi-permeable polymeric enclosures or membranes that allow the release of the protein product(s) but prevent the destruction of the cells by the patient's immune system or by other detrimental factors from the surrounding tissues.

As discussed herein, it can be desirable to treat isolated cell populations (such as stem cells, lymphocytes, red blood cells, chondrocytes, neurons, and the like) with one or more Cdk4 inhibitors of the invention. This can be accomplished by exposing the isolated cells to the Cdk4 inhibitors of the invention directly, in a form that is permeable to the cell membrane.

Additional embodiments of the present invention relate to cells and methods (e.g., homologous recombination and/or other recombinant production methods) for both the in vitro production of therapeutic polypeptides and for the production and delivery of therapeutic polypeptides by gene therapy or cell therapy. Homologous and other recombination methods can be used to modify a cell that contains a normally transcriptionally-silent Cdk4 inhibitory gene, or an under-expressed gene, and thereby produce a cell that expresses therapeutically efficacious amounts of Cdk4 inhibitory polypeptides. Cdk4 inhibitory polypeptides include, but are not limited to, dominant-negative mutants and endogenous polypeptides that downregulate Cdk4 expression and/or activity, such as angiotensin II type II (AT(2)) receptor subtype (Gingras et al., 2003, Oncogene 22:2633-42).

Homologous recombination is a technique originally developed for targeting genes to induce or correct mutations in transcriptionally active genes. See, Kucherlapati, 1989, Prog. in Nucl. Acid Res. & Mol. Biol. 36:301. The basic technique was developed as a method for introducing specific mutations into specific regions of the mammalian genome (Thomas et al., 1986, Cell 44:419-28; Thomas and Capecchi, 1987, Cell 51:503-12; Doetschman et al., 1988, Proc. Natl. Acad. Sci. U.S.A. 85:8583-87) or to correct specific mutations within defective genes (Doetschman et al., 1987, Nature 330:576-78). Exemplary homologous recombination techniques are described in U.S. Pat. No. 5,272,071; European Patent Nos. 9193051 and 505500; PCT/US90/07642, and PCT Pub No. WO 91/09955.

Through homologous recombination, the DNA sequence to be inserted into the genome can be directed to a specific region of the gene of interest by attaching it to targeting DNA. The targeting DNA is a nucleotide sequence that is complementary (homologous) to a region of the genomic DNA. Small pieces of targeting DNA that are complementary to a specific region of the genome are put in contact with the parental strand during the DNA replication process. It is a general property of DNA that has been inserted into a cell to hybridize, and therefore, recombine with other pieces of endogenous DNA through shared homologous regions. If this complementary strand is attached to an oligonucleotide that contains a mutation or a different sequence or an additional nucleotide, it too is incorporated into the newly synthesized strand as a result of the recombination. As a result of the proofreading function, it is possible for the new sequence of DNA to serve as the template. Thus, the transferred DNA is incorporated into the genome.

Attached to these pieces of targeting DNA are regions of DNA that may interact with or control the expression of a Cdk4 inhibitory polypeptide, e.g., flanking sequences. For example, a promoter/enhancer element, a suppressor, or an exogenous transcription modulatory element is inserted in the genome of the intended host cell in proximity and orientation sufficient to influence the transcription of DNA encoding the desired Cdk4 inhibitory polypeptide. The control element controls a portion of the DNA present in the host cell genome. Thus, the expression of the desired Cdk4 inhibitory polypeptide can be achieved not by transfection of DNA that encodes the Cdk4 inhibitory gene itself, but rather by the use of targeting DNA (containing regions of homology with the endogenous gene of interest) coupled with DNA regulatory segments that provide the endogenous gene sequence with recognizable signals for transcription of a Cdk4 inhibitory gene.

In an exemplary method, the expression of a desired targeted gene in a cell (i.e., a desired endogenous cellular gene) is altered via homologous recombination into the cellular genome at a preselected site, by the introduction of DNA that includes at least a regulatory sequence, an exon, and a splice donor site. These components are introduced into the chromosomal (genomic) DNA in such a manner that this, in effect, results in the production of a new transcription unit (in which the regulatory sequence, the exon, and the splice donor site present in the DNA construct are operatively linked to the endogenous gene). As a result of the introduction of these components into the chromosomal DNA, the expression of the desired endogenous gene is altered.

Altered gene expression, as described herein, encompasses activating (or causing to be expressed) a gene which is normally silent (unexpressed) in the cell as obtained, as well as increasing the expression of a gene which is not expressed at physiologically significant levels in the cell as obtained. The embodiments further encompass changing the pattern of regulation or induction such that it is different from the pattern of regulation or induction that occurs in the cell as obtained, and reducing (including eliminating) the expression of a gene which is expressed in the cell as obtained.

One method by which homologous recombination can be used to increase, or cause, Cdk4 inhibitory polypeptide production from a cell's endogenous Cdk4 inhibitory gene involves first using homologous recombination to place a recombination sequence from a site-specific recombination system (e.g., Cre/loxP, FLP/FRT) (Sauer, 1994, Curr. Opin. Biotechnol., 5:521-27; Sauer, 1993, Methods Enzymol., 225:890-900) upstream of (i.e., 5′ to) the cell's endogenous genomic Cdk4 inhibitory polypeptide coding region. A plasmid containing a recombination site homologous to the site that was placed just upstream of the genomic Cdk4 inhibitory polypeptide coding region is introduced into the modified cell line along with the appropriate recombinase enzyme. This recombinase causes the plasmid to integrate, via the plasmid's recombination site, into the recombination site located just upstream of the genomic Cdk4 inhibitory polypeptide coding region in the cell line (Baubonis and Sauer, 1993, Nucleic Acids Res. 21:2025-29; O'Gorman et al., 1991, Science 251:1351-55). Any flanking sequences known to increase transcription (e.g., enhancer/promoter, intron, translational enhancer), if properly positioned in this plasmid, would integrate in such a manner as to create a new or modified transcriptional unit resulting in de novo or increased Cdk4 inhibitory polypeptide production from the cell's endogenous Cdk4 inhibitory gene.

A two-recombination-site cell line can also be used in a method of the invention. For example, a site-specific recombination sequence can be placed upstream of a cell's endogenous genomic Cdk4 inhibitory polypeptide coding region, while a second recombination site can be introduced elsewhere in the cell line's genome using homologous recombination. The appropriate recombinase enzyme is then introduced into the two-recombination-site cell line, causing a recombination event (deletion, inversion, and translocation) (Sauer, 1994, Curr. Opin. Biotechnol., 5:521-27; Sauer, 1993, Methods Enzymol., 225:890-900) that would create a new or modified transcriptional unit resulting in de novo or increased Cdk4 inhibitory polypeptide production from the cell's endogenous Cdk4 inhibitory gene.

An additional approach for increasing, or causing, the expression of Cdk4 inhibitory polypeptide from a cell's endogenous Cdk4 inhibitory gene involves increasing, or causing, the expression of a gene or genes (e.g., transcription factors) and/or decreasing the expression of a gene or genes (e.g., transcriptional repressors) in a manner which results in de novo or increased Cdk4 inhibitory polypeptide production from the cell's endogenous Cdk4 inhibitory gene. This method includes the introduction of a non-naturally occurring polypeptide (e.g., a polypeptide comprising a site specific DNA binding domain fused to a transcriptional factor domain) into the cell such that de novo or increased Cdk4 inhibitory polypeptide production from the cell's endogenous Cdk4 inhibitory gene results.

The present invention further relates to DNA constructs useful in the method of altering expression of a target gene. In certain embodiments, the exemplary DNA constructs comprise: (a) one or more targeting sequences, (b) a regulatory sequence, (c) an exon, and (d) an unpaired splice-donor site. The targeting sequence in the DNA construct directs the integration of elements (a)-(d) into a target gene in a cell such that the elements (b)-(d) are operatively linked to sequences of the endogenous target gene. In another embodiment, the DNA constructs comprise: (a) one or more targeting sequences, (b) a regulatory sequence, (c) an exon, (d) a splice-donor site, (e) an intron, and (f) a splice-acceptor site, wherein the targeting sequence directs the integration of elements (a)-(f) such that the elements of (b)-(f) are operatively linked to the endogenous gene. The targeting sequence is homologous to the preselected site in the cellular chromosomal DNA with which homologous recombination is to occur. In the construct, the exon is generally 3′ of the regulatory sequence and the splice-donor site is 3′ of the exon.

If the sequence of a particular gene is known, such as the nucleic acid sequence of Cdk4 inhibitory polypeptide presented herein, a DNA fragment complementary to a selected region of the gene can be synthesized or otherwise obtained, such as by appropriate restriction of the native DNA at specific recognition sites bounding the region of interest. Such fragments serve as a targeting sequence upon insertion into the cell and hybridize to a homologous region within the genome. If this hybridization occurs during DNA replication, this DNA fragment, and any additional sequence attached thereto, will act as an Okazaki fragment and be incorporated into the newly synthesized daughter strand of DNA. The present invention, therefore, includes nucleotides encoding a Cdk4 inhibitory polypeptide, which nucleotides may be used as targeting sequences.

Cdk4 inhibitory polypeptide cell therapy, e.g., the implantation of cells producing Cdk4 inhibitory polypeptides, is also contemplated. This embodiment involves implanting cells capable of synthesizing and secreting a biologically active form of Cdk4 inhibitory polypeptide. Such Cdk4 inhibitory polypeptide-producing cells can be cells that are natural producers of Cdk4 inhibitory polypeptides or may be recombinant cells whose ability to produce Cdk4 inhibitory polypeptides has been augmented by transformation with a gene encoding the desired Cdk4 inhibitory polypeptide or with a gene augmenting the expression of Cdk4 inhibitory polypeptide. Such a modification may be accomplished by means of a vector suitable for delivering the gene as well as promoting its expression and secretion. In order to minimize a potential immunological reaction in patients being administered a Cdk4 inhibitory polypeptide, as may occur with the administration of a polypeptide of a foreign species, it is preferred that the natural cells producing Cdk4 inhibitory polypeptide be of human origin and produce human Cdk4 inhibitory polypeptide. Likewise, it is preferred that the recombinant cells producing Cdk4 inhibitory polypeptide be transformed with an expression vector containing a gene encoding a human Cdk4 inhibitory polypeptide.

Implanted cells may be encapsulated to avoid the infiltration of surrounding tissue. Human or non-human animal cells may be implanted in patients in biocompatible, semipermeable polymeric enclosures or membranes that allow the release of Cdk4 inhibitory polypeptide, but that prevent the destruction of the cells by the patient's immune system or by other detrimental factors from the surrounding tissue. Alternatively, the patient's own cells, transformed to produce Cdk4 inhibitory polypeptides ex vivo, may be implanted directly into the patient without such encapsulation.

Techniques for the encapsulation of living cells are known in the art, and the preparation of the encapsulated cells and their implantation in patients may be routinely accomplished. For example, Baetge et al. (PCT Pub. No. WO 95/05452 and PCT/US94/09299) describe membrane capsules containing genetically engineered cells for the effective delivery of biologically active molecules. The capsules are biocompatible and are easily retrievable. The capsules encapsulate cells transfected with recombinant DNA molecules comprising DNA sequences coding for biologically active molecules operatively linked to promoters that are not subject to down-regulation in vivo upon implantation into a mammalian host. The devices provide for the delivery of the molecules from living cells to specific sites within a recipient. In addition, see U.S. Pat. Nos. 4,892,538; 5,011,472; and 5,106,627. A system for encapsulating living cells is described in PCT Pub. No. WO 91/10425 (Aebischer et al.). See also, PCT Pub. No. WO 91/10470 (Aebischer et al.); Winn et al., 1991, Exper. Neurol. 113:322-29; Aebischer et al., 1991, Exper. Neurol. 111:269-75; and Tresco et al., 1992, ASAIO 38:17-23.

In vivo and in vitro gene therapy delivery of Cdk4 inhibitory polypeptides is also envisioned. One example of a gene therapy technique is to use the Cdk4 inhibitory gene (either genomic DNA, cDNA, and/or synthetic DNA) encoding a Cdk4 inhibitory polypeptide that may be operably linked to a constitutive or inducible promoter to form a “gene therapy DNA construct.” The promoter can be homologous or heterologous to the endogenous Cdk4 inhibitory gene, provided that it is active in the cell or tissue type into which the construct will be inserted. Other components of the gene therapy DNA construct can optionally include DNA molecules designed for site-specific integration (e.g., endogenous sequences useful for homologous recombination), tissue-specific promoters, enhancers or silencers, DNA molecules capable of providing a selective advantage over the parent cell, DNA molecules useful as labels to identify transformed cells, negative selection systems, cell specific binding agents (as, for example, for cell targeting), cell-specific internalization factors, transcription factors enhancing expression from a vector, and factors enabling vector production.

A gene therapy DNA construct can then be introduced into cells (either ex vivo or in vivo) using viral or non-viral vectors. One means for introducing the gene therapy DNA construct is by means of viral vectors as described herein. Certain vectors, such as retroviral vectors, will deliver the DNA construct to the chromosomal DNA of the cells, and the gene can integrate into the chromosomal DNA. Other vectors will function as episomes, and the gene therapy DNA construct will remain in the cytoplasm.

In yet other embodiments, regulatory elements can be included for the controlled expression of the Cdk4 inhibitory gene in the target cell. Such elements are turned on in response to an appropriate effector. In this way, a therapeutic polypeptide can be expressed when desired. One conventional control means involves the use of small molecule dimerizers or rapalogs to dimerize chimeric proteins which contain a small molecule-binding domain and a domain capable of initiating a biological process, such as a DNA-binding protein or transcriptional activation protein (see PCT Pub. Nos. WO 96/41865, WO 97/31898, and WO 97/31899). The dimerization of the proteins can be used to initiate transcription of the transgene.

An alternative regulation technology uses a method of storing proteins expressed from the gene of interest inside the cell as an aggregate or cluster. The gene of interest is expressed as a fusion protein that includes a conditional aggregation domain that results in the retention of the aggregated protein in the endoplasmic reticulum. The stored proteins are stable and inactive inside the cell. The proteins can be released, however, by administering a drug (e.g., small molecule ligand) that removes the conditional aggregation domain and thereby specifically breaks apart the aggregates or clusters so that the proteins can be secreted from the cell. See Aridor et al., 2000, Science 287:816-17 and Rivera et al., 2000, Science 287:826-30.

Other suitable control means or gene switches include, but are not limited to, the systems described herein. Mifepristone (RU486) is used as a progesterone antagonist. The binding of a modified progesterone receptor ligand-binding domain to the progesterone antagonist activates transcription by forming a dimmer of two transcription factors that then pass into the nucleus to bind DNA. The ligand-binding domain is modified to eliminate the ability of the receptor to bind to the natural ligand. The modified steroid hormone receptor system is further described in U.S. Pat. No. 5,364,791 and PCT Pub. Nos. WO 96/40911 and WO 97/10337.

Yet another control system uses ecdysone (a fruit fly steroid hormone) which binds to and activates an ecdysone receptor (cytoplasmic receptor). The receptor then Tran locates to the nucleus to bind a specific DNA response element (promoter from ecdysone-responsive gene). The ecdysone receptor includes a transactivation domain, DNA-binding domain, and ligand-binding domain to initiate transcription. The ecdysone system is further described in U.S. Pat. No. 5,514,578 and PCT Pub. Nos. WO 97/38117, WO 96/37609, and WO 93/03162.

Another control means uses a positive tetracycline-controllable transactivator. This system involves a mutated Tet repressor protein DNA-binding domain (mutated tet R-4 amino acid changes which resulted in a reverse tetracycline-regulated transactivator protein, i.e., it binds to a tet operator in the presence of tetracycline) linked to a polypeptide which activates transcription. Such systems are described in U.S. Pat. Nos. 5,464,758, 5,650,298, and 5,654,168.

Additional expression control systems and nucleic acid constructs are described in U.S. Pat. Nos. 5,741,679 and 5,834,186, to Involver Laboratories Inc.

In vivo gene therapy may be accomplished by introducing a nucleic acid molecule of the invention into cells via local injection or by other appropriate viral or non-viral delivery vectors. Hefty, 1994, Neurobiology 25:1418-35. For example, a nucleic acid molecule of the invention can be contained in an adenoma-associated virus (AAV) vector for delivery to the targeted cells (see, e.g., Johnson, PCT Pub. No. WO 95/34670; PCT App. No. PCT/US95/07178). The recombinant AAV genome typically contains AAV inverted terminal repeats flanking a nucleic acid molecule of the invention operably linked to functional promoter and polyadenylation sequences.

Alternative suitable viral vectors include, but are not limited to, retrovirus, adenovirus, herpes simplex virus, lentivirus, hepatitis virus, parvovirus, papovavirus, poxvirus, alphavirus, corona virus, rhabdovirus, paramyxovirus, and papilloma virus vectors. U.S. Pat. No. 5,672,344 describes an in vivo viral-mediated gene transfer system involving a recombinant neurotrophic HSV-1 vector. U.S. Pat. No. 5,399,346 provides examples of a process for providing a patient with a therapeutic protein by the delivery of human cells that have been treated in vitro to insert a DNA segment encoding a therapeutic protein. Additional methods and materials for the practice of gene therapy techniques are described in U.S. Pat. Nos. 5,631,236 (involving adenoviral vectors), 5,672,510 (involving retroviral vectors), 5,635,399 (involving retroviral vectors expressing cytokines).

Nonviral delivery methods include, but are not limited to, liposome-mediated transfer, naked DNA delivery (direct injection), receptor-mediated transfer (ligand-DNA complex), electroporation, calcium phosphate precipitation, and microparticle bombardment (e.g., gene gun). Gene therapy materials and methods may also include inducible promoters, tissue-specific enhancer-promoters, DNA sequences designed for site-specific integration, DNA sequences capable of providing a selective advantage over the parent cell, labels to identify transformed cells, negative selection systems and expression control systems (safety measures), cell-specific binding agents (for cell targeting), cell-specific internalization factors, and transcription factors to enhance expression by a vector as well as methods of vector manufacture. Such additional methods and materials for the practice of gene therapy techniques are described in U.S. Pat. Nos. 4,970,154 (involving electroporation techniques), 5,679,559 (describing a lipoprotein-containing system for gene delivery), 5,676,954 (involving liposome carriers), 5,593,875 (describing methods for calcium phosphate transfection), and 4,945,050 (describing a process wherein biologically active particles are propelled at cells at a speed whereby the particles penetrate the surface of the cells and become incorporated into the interior of the cells), and PCT Pub. No. WO 96/40958 (involving nuclear ligands).

It is also contemplated that Cdk4 gene therapy or cell therapy can further include the delivery of one or more additional polypeptide(s) in the same or a different cell(s). Such cells can be separately introduced into the patient, or the cells can be contained in a single implantable device, such as the encapsulating membrane described above, or the cells can be separately modified by means of viral vectors.

Gene therapy also can be used to decrease Cdk4 polypeptide expression by modifying the nucleotide sequence of the endogenous promoter. Such modification is typically accomplished via homologous recombination methods. For example, a DNA molecule containing all or a portion of the promoter of the Cdk4 gene selected for inactivation can be engineered to remove and/or replace pieces of the promoter that regulate transcription. For example, the TATA box and/or the binding site of a transcriptional activator of the promoter can be deleted using standard molecular biology techniques; such deletion can inhibit promoter activity thereby repressing the transcription of the corresponding Cdk4 gene. The deletion of the TATA box or the transcription activator binding site in the promoter may be accomplished by generating a DNA construct comprising all or the relevant portion of the Cdk4 polypeptide promoter (from the same or a related species as the Cdk4 gene to be regulated) in which one or more of the TATA box and/or transcriptional activator binding site nucleotides are mutated via substitution, deletion and/or insertion of one or more nucleotides. As a result, the TATA box and/or activator binding site has decreased activity or is rendered completely inactive. This construct, which also will typically contain at least about 500 bases of DNA that correspond to the native (endogenous) 5′ and 3′ DNA sequences adjacent to the promoter segment that has been modified, may be introduced into the appropriate cells (either ex vivo or in vivo) either directly or via a viral vector as described herein. Typically, the integration of the construct into the genomic DNA of the cells will be via homologous recombination, where the 5′ and 3′ DNA sequences in the promoter construct can serve to help integrate the modified promoter region via hybridization to the endogenous chromosomal DNA.

Alternatively, certain siRNA molecules of the invention can be expressed within cells from eukaryotic promoters (see for example, Izant and Weintraub, 1985, Science 229:345; McGarry and Lindquist, 1986, Proc. Natl. Acad. Sci., USA 83:399; Scanlon et al., 1991, Proc. Natl. Acad. Sci. USA 88:10591-5; Kashani-Sabet et al., 1992, Antisense Res. Dev. 2.3-15; Dropulic et al., 1992, J. Virol. 66:1432-41; Weerasinghe et al., 1991, J. Virol. 65:5531-4; Ojwang et al., 1992, Proc. Natl. Acad. Sci. USA 89:10802-6; Chen et al., 1992, Nucleic Acids Res. 20:4581-9; Sarver et al., 1990, Science 247:1222-1225; Thompson et al., 1995, Nucleic Acids Res. 23:2259; Good et al., 1997, Gene Therapy 4: 45. Those skilled in the art will recognize that any nucleic acid can be expressed in eukaryotic cells using the appropriate DNA/RNA vector. The activity of such nucleic acids can be augmented by their release from the primary transcript by an enzymatic nucleic acid (Draper et al., PCT WO 93/23569, and Sullivan et al., PCT WO 94/02595; Ohkawa et al., 1992, Nucleic Acids Symp. Ser. 27:15-6; Taira et al., 1991, Nucleic Acids Res. 19:5125-30; Ventura et al., 1993, Nucleic Acids Res. 21:3249-55; Chowrira et al., 1994, J. Biol. Chem. 269:25856.

In another aspect of the invention, RNA molecules of the present invention can be expressed from transcription units (see for example, Couture et al., 1996, TIG 12:510) inserted into DNA or RNA vectors. The recombinant vectors can be DNA plasmids or viral vectors. siRNA expressing viral vectors can be constructed based on, but not limited to, adeno-associated virus, retrovirus, adenovirus, or alphavirus. In another embodiment, pol III based constructs are used to express nucleic acid molecules of the invention (see for example, Thompson, U.S. Pat. Nos. 5,902,880 and 6,146,886). The recombinant vectors capable of expressing the siRNA molecules can be delivered as described above, and persist in target cells. Alternatively, viral vectors can be used that provide for transient expression of nucleic acid molecules. Such vectors can be repeatedly administered as necessary. Once expressed, the siRNA molecule interacts with the target mRNA and generates an RNAi response. Delivery of siRNA molecule expressing vectors can be systemic, such as by intravenous or intramuscular administration, by administration to target cells ex-planted from a subject followed by reintroduction into the subject, or by any other means that would allow for introduction into the desired target cell (for a review, see Couture et al., 1996, TIG. 12:510).

In one embodiment, the invention provides an expression vector comprising a nucleic acid sequence encoding at least one siRNA molecule of the invention. The expression vector can encode one or both strands of a siRNA duplex, or a single self-complementary strand that self hybridizes into an siRNA duplex. The nucleic acid sequences encoding the siRNA molecules can be operably linked in a manner that allows expression of the siRNA molecule (see for example, Paul et al., 2002, Nature Biotechnology 19:505; Miyagishi and Taira, 2002, Nature Biotechnology 19:497; Lee et al., 2002, Nature Biotechnology 19:500; and Novina et al., 2002, Nature Medicine, online publication June 3). The term “operably linked” is used herein to refer to an arrangement of flanking sequences wherein the flanking sequences so described are configured or assembled so as to perform their usual function. Thus, a flanking sequence operably linked to a coding sequence may be capable of effecting the replication, transcription and/or translation of the coding sequence. For example, a coding sequence is operably linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence and the promoter sequence can still be considered “operably linked” to the coding sequence.

In another aspect, the invention provides an expression vector comprising: a) a transcription initiation region (e.g., eukaryotic pol I, II or III initiation region); b) a transcription termination region (e.g., eukaryotic pol I, II or III termination region); and c) a nucleic acid sequence encoding at least one of the siRNA molecules of the invention; wherein said sequence is operably linked to said initiation region and said termination region, in a manner that allows expression and/or delivery of the siRNA molecule. The vector can optionally include an open reading frame (ORF) for a protein operably linked on the 5′ side or the 3′-side of the sequence encoding the siRNA of the invention; and/or an intron (intervening sequences).

Transcription of the siRNA molecule sequences can be driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA 87:6743-7; Gao and Huang 1993, Nucleic Acids Res. 21:2867-72; Lieber et al., 1993, Methods Enzymol. 217:47-66; Zhou et al. 1990, Mol. Cell. Biol. 10:4529-37). Several investigators have demonstrated that nucleic acid molecules expressed from such promoters can function in mammalian cells (e.g. Kashani-Sabet et al., 1992, Antisense Res. Dev. 2:3-15; Ojwang et al., 1992, Proc. Natl. Acad. Sci, USA 89:10802-6; Chen et al., 1992, Nucleic Acids Res. 20:4581-9; Yu et al., 1993, Proc. Natl. Acad. Sci. USA 90:6340-4; L'Huillier et al., 1992, EMBO J. 11:4411-8; Lisziewicz et al., 1993, Proc. Natl. Acad. Sci. U.S.A 90:8000-4; Thompson et al., 1995, Nucleic Acids Res. 23:2259; Sullenger and Cech, 1993, Science 262:1566). More specifically, transcription units such as the ones derived from genes encoding U6 small nuclear (snRNA), transfer RNA (tRNA) and adenovirus VA RNA are useful in generating high concentrations of desired RNA molecules such as siRNA in cells (Thompson et al., 1995, Nucleic Acids Res. 23:2259; Couture et al., 1996, TIG 12:510; Noonberg et al., 1994, Nucleic Acid Res. 22:2830; Noonberg et al., U.S. Pat. No. 5,624,803; Good et al., 1997, Gene Ther. 4:45; Beigelman et al., International PCT Publication No. WO 96/18736. The above siRNA transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture et al., 1996, TIG 12:510).

In another embodiment, the invention provides an expression vector comprising a nucleic acid sequence encoding at least one of the siRNA molecules of the invention, in a manner that allows expression of that siRNA molecule. In a particular embodiment, the expression vector comprises: a) a transcription initiation region; b) a transcription termination region; and c) a nucleic acid sequence encoding at least one strand of the siRNA molecule; wherein the sequence is operably linked to the initiation region and the termination region, in a manner that allows expression and/or delivery of the siRNA molecule.

In another embodiment the expression vector comprises: a) a transcription initiation region; b) a transcription termination region; c) an open reading frame; and d) a nucleic acid sequence encoding at least one strand of a siRNA molecule, wherein the sequence is operably linked to the 3′-end of the open reading frame; and wherein the sequence is operably linked to the initiation region, the open reading frame and the termination region, in a manner that allows expression and/or delivery of the siRNA molecule.

In yet another embodiment the expression vector comprises: a) a transcription initiation region; b) a transcription termination region; c) an intron; and d) a nucleic acid sequence encoding at least one siRNA molecule; wherein the sequence is operably linked to the initiation region, the intron and the termination region, in a manner which allows expression and/or delivery of the nucleic acid molecule.

In another embodiment, the expression vector comprises: a) a transcription initiation region; b) a transcription termination region; c) an intron; d) an open reading frame; and e) a nucleic acid sequence encoding at least one strand of a siRNA molecule, wherein the sequence is operably linked to the 3′-end of the open reading frame; and wherein the sequence is operably linked to the initiation region, the intron, the open reading frame and the termination region, in a manner which allows expression and/or delivery of the siRNA molecule.

Conventional techniques were used herein for recombinant DNA, oligonucleotide synthesis, and tissue culture and transformation (e.g., electroporation, lipofection). Enzymatic reactions and purification techniques were performed according to manufacturers' specifications or as commonly accomplished in the art or as described herein. The techniques and procedures were generally performed according to conventional methods well known in the art and as described in various general and more specific references that are cited and discussed throughout the present specification. See e.g., Sambrook et al., 2001, MOLECULAR CLONING: A LABORATORY MANUAL, 3d ed., Cold Spring Harbor Laboratory Press, Cold Spring Harbor, N.Y., which is incorporated herein by reference for any purpose. Unless specific definitions are provided, the nomenclature utilized in connection with, and the laboratory procedures and techniques of, molecular biology, genetic engineering, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

Unless otherwise required by context, singular terms shall include pluralities and plural terms shall include the singular.

The following examples, including the experiments conducted and results achieved are provided for illustrative purposes only and are not to be construed as limiting the invention.

### EXAMPLES

### Example 1

### Cdk4-Null MEF are Resistant to Transformation in Response to Ras Activation and p53 Inhibition

The effect of Cdk4 disruption on transformation potential was examined using Cdk4^{+/+} and Cdk4^{-/-} mouse embryonic fibroblasts (MEF) from embryos obtained from intercross breeding of Cdk4^{+/-} mice (Tsutsui et al., 1999, Mol. Cell Biol. 19:7011-7019). A targeted null mutation of the Cdk4 gene, Cdk4^{tm1/Kiyo}, was created by homologous recombination in mouse embryonic stem cells, and mice with germline transmission of this mutation were bred in the recombinant C57BL/6×129/svj strain background, as described (Tsutsui et al., 1999, Mol. Cell Biol. 19:7011-7019). MEF were prepared from day 12.5 mouse embryos and cultured in the Dulbecco's modified minimum essential medium supplemented with 2 mM glutamine, 100 U/ml penicillin and streptomycin, and 10% fetal bovine serum (FBS) (Life Technology, Grand Island, N.Y.), as described previously (Tsutsui et al., 1999, Mol. Cell Biol. 19:7011-7019). MEF dispersed from each embryo using 0.25% trypsin solution containing 0.53 mM EDTA were cultured in a 100-mm culture dish (passage 1). Cells were then maintained using a 3T3 protocol (3×10⁵ cells per 60-mm culture dish passaged every 3 days). The population doubling level during each passage was calculated according to the formula log(final cell number/3×10⁵)/log2.

Cells at early passages (passage 3-4) were infected with a retrovirus for expression of oncogenic H-Ras^{Val12} and a dominant negative p53 mutant (DNp53), previously described as GSE56 (Ossovskaya et al., 1996, Proc. Natl. Acad. Sci. U.S.A. 93:10309-10314). DNp53 encoded amino acids 275-368 of p53, and suppressed p53 activity, presumably by interfering with oligomerization of the protein. The Phoenix ecotropic virus packaging cells were obtained from the American Tissue Culture Collection (ATCC) with permission of Gary P. Nolan (Stanford University). The pBabehygro vector for expression of H-Ras^{Val12} was described previously (Serrano et al., 1996, Cell 85:27-37). The LXSN vector for coexpression of DNp53 (GSE56) (Ossovskaya et al., 1996, Proc. Natl. Acad. Sci. U.S.A. 93:10309-10314) and H-Ras^{Val12} was constructed using the internal ribosomal entry site. Phoenix cells were transfected with vectors using the SuperFect transfection reagent (Qiagen, Santa Clara, Calif.), and culture supernatants containing infectious retrovirus were harvested 48 hr posttransfection, as described previously (Pear et al., 1993, Proc. Natl. Acad. Sci. U.S.A. 90:8392-8396). Virus-containing supernatants were pooled and filtered through 0.45-mm membrane. Infections of exponentially growing MEF were performed with 1.5 ml of various dilutions of virus-containing supernatant supplemented with 10 μg/ml polybrene (Sigma, St. Louis, Mo.) for each 60-mm culture dish. The dilutions of the H-Ras^{Val12} and DNp53+H-Ras^{Val12} used were determined according to the numbers of transformed foci developed in Cdk4^{+/+} Ink4a/Arf^{-/-} and wild-type MEF, respectively, in pilot experiments. After 3 hr, cells were rinsed and 5 ml fresh medium was added.

For transformed focus formation, MEF were cultured in complete medium with 5% FBS without splitting, for 14-21 days after retrovirus infection. Medium was changed every 3 days. Confluent monolayer cultures with foci were rinsed with phosphate buffered saline (PBS), and stained with 4 mg/ml crystal violet in 10% methanol. Unstained foci of morphologically transformed cells were picked under a phase microscope (Nikon), subcloned by limited trypsinization, and expanded for the tumorigenicity assay. For colony formation in soft agar, MEF at 48 hrs post-viral infection were trypsinized, counted and inoculated at 10⁶ cells per 60-mm dish in 0.3% Noble agar in DMEM supplemented with 10% FBS. Colonies were scored 21-28 days later. When cells isolated from foci were tested for anchorage independent growth, 2×10⁴ cells were inoculated per dish in the Noble agar medium.

Under standard culture conditions with 10% fetal bovine serum, Cdk4^{-/-} MEF proliferated at rates indistinguishable from those of Cdk4^{+/+} MEF, as demonstrated previously (Tsutsui et al., 1999, Mol. Cell Biol. 19:7011-7019). Following retroviral transduction of H-Ras^{Val12}, with or without DNp53, cells were cultured for 21 days without splitting and then stained to visualize transformed foci (FIG. 1A). Strikingly, the numbers of foci developed in Cdk4^{-/-} MEF cultures expressing H-Ras^{Val12} and DNp53 were 95% reduced, relative to those in Cdk4^{+/+} cultures. Retroviral transduction of H-Ras^{Val12} alone or DNp53 alone did not result in focus formation in either Cdk4^{+/+} or Cdk4^{-/-} MEF. Immunoblotting confirmed that the levels of Ras expression were comparable in Cdk4^{+/+} and Cdk4^{-/-} cells. Retroviral transduction of Cdk4 prior to transduction of H-Ras^{Val12} and DNp53 restored foci formation (FIG. 1B), which confirmed that the absence of Cdk4 was responsible for the inhibition of foci formation.

Anchorage-independent growth was examined by plating MEF in soft agar following retroviral transduction (FIGS. 2A and 2B). Whereas Cdk4^{+/+} MEF expressing H-Ras^{Val12} and DNp53 efficiently developed colonies in soft agar, Cdk4^{-/-} MEF did not form detectable colonies under the same conditions. MEF expressing H-Ras^{Val12} alone or DNp53 alone formed no colonies regardless of the Cdk4 genotype, as expected. These data suggested that Cdk4 disruption inhibited cellular transformation induced by Ras activation and p53 inhibition.

### Example 2

### Cdk4^{-/-} Ink4a/Arf^{-/-} MEF are Resistant to Ras-Induced Transformation

The effect of Cdk4 deficiency on Ras-mediated transformation was examined using Cdk4^{-/-} Ink4a/Arf^{-/-} and Cdk4^{+/+} Ink4a/Arf^{-/-} MEF, which were prepared by crossing Cdk4-null mice and mice with deletion of the exons 2 and 3 of the Ink4a/Arf locus (Serrano et al., 1996, Cell 85:27-37). Cells at early passage were infected with retrovirus for H-Ras^{Val12} or control virus, and then cultured for 17 days without splitting. Cdk4^{+/+} Ink4a/Arf^{-/-} MEF efficiently developed transformed foci upon retroviral transduction of H-Ras (FIGS. 3A and 3B), as previously demonstrated (Serrano et al., 1996, Cell 85:27-37). In contrast, Cdk4^{-/-} Ink4a/Arf^{-/-} MEF expressing H-Ras^{Val12} poorly formed foci, showing 93% reduction in number. No colonies grew when Cdk4^{-/-} Ink4a/Arf^{-/-} MEF were inoculated in soft agar following H-Ras^{Val12} transduction, whereas Cdk4_{+/+} Ink4a/Arf^{-/-} MEF readily developed colonies. These observations suggested that Cdk4 played a major role in transformation of MEF induced by Ras activation in the Ink4a/Arf-null background.

### Example 3

### Cdk4-Null Cells Isolated from Foci are not Tumorigenic In Vivo

To determine whether Cdk4-null cells that formed foci were tumorigenic in vivo, Cdk4^{+/+} and Cdk4^{-/-} MEF clones were injected into athymic mice. The Cdk4^{+/+} and Cdk4^{-/-} MEF clones were isolated from foci induced by HRasVal12 and DNp53 retroviral transduction. Cdk4^{-/-} clones exhibited slower proliferation in culture, compared with Cdk4^{+/+} clones. Five independent clones with each genotype were tested (FIGS. 4A and 4B).

For in vivo tumor formation, 10⁶ cells isolated and expanded from foci were injected into flanks of 7-week-old athymic mice (National Cancer Institute, Frederick, Md.). Two mice were used for each clone. Tumor formation was scored every week, and diameters of palpable tumors were recorded. At 21 days post-injection, all five Cdk4^{+/+} clones displayed tumor growth, with diameters of 1.7±0.5 cm (mean ±SEM). In contrast, none of five Cdk4^{-/-} clones developed detectable tumors in athymic mice during a 6-week monitoring period (FIG. 4A)

The in vivo tumorigenicity of Cdk4^{+/+} Ink4a/Arf^{-/-} and Cdk4^{-/-} Ink4a/Arf^{-/-} MEF clones was also examined by injecting the MEF into athymic mice. The Cdk4^{+/+} Ink4a/Arf^{-/-} and Cdk4^{-/-} Ink4a/Arf^{-/-} MEF clones were isolated and expanded from foci induced by H-Ras^{Val12}. Cdk4^{-/-} Ink4a/Arf^{-/-} clones did not develop detectable tumors in athymic mice, whereas mice injected with Cdk4^{+/+} Ink4a/Arf^{-/-} clones readily displayed large tumors (FIG. 4B). These data suggested that Cdk4 disruption abrogated tumorigenicity of MEF induced by Ras activation with p53 inhibition or Ink4a/Arf disruption.

### Example 4

### Cdk4 Deficiency Leads Ink4a/Arf-Null MEF to Senescence

It has been well established that MEF lacking p53 or Arf are immortal in culture, devoid of “culture shock”-induced senescence, and are readily transformed by activated Ras (Serrano et al., 1997, Cell 88:593-602; Kamijo et al., 1997, Cell 91:649-659). Immortalization is a process required for the multi-step oncogenic transformation. To further investigate the mechanism of the transformation-inhibitory action of Cdk4 disruption, Cdk4^{-/-} Ink4a/Arf^{-/-} MEF were examined for an immortal phenotype similar to Cdk4^{+/+} Ink4a/Arf^{-/-} MEF.

Cells at a late passage (passage 11) were inoculated at a low density (1,000 cells per dish), and cultured for 10 days to score colonies derived from isolated cells (FIG. 5A). Cdk4^{+/+} Ink4a/Arf^{-/-} MEF formed >200 large colonies, indicating clonogenic proliferation with high plating efficiency. In contrast, Cdk4^{-/-} Ink4a/Arf^{-/-} MEF exhibited very few colonies. These observations suggested that Cdk4 disruption impairs clonogenic proliferation of Ink4a/Arf-null cells.

The proliferative life spans of Cdk4^{+/+} Ink4a/Arf^{-/-} and Cdk4^{-/-} Ink4a/Arf^{-/-} MEF were also examined by monitoring population doublings during continuous culture according to the 3T3 protocol (FIG. 5B). Cdk4^{+/+} Ink4a/Arf^{-/-} escaped from senescence, as expected. In contrast, Cdk4^{-/-} Ink4a/Arf^{-/-} MEF underwent growth arrest after 22-24 population doublings similar to wild-type MEF.

Cdk4^{-/-} Ink4a/Arf^{-/-} cells at late passages displayed a flat enlarged morphology and senescence-associated β-galactosidase (SA-β-gal) activity (FIG. 5C), which are characteristic of cellular senescence (Dimri et al., 1995, Proc. Natl. Acad. Sci. U.S.A. 92:9363-9367). SA-β-gal activity at pH 6.0 was assayed, as described previously (Dimri et al., 1995, Proc. Natl. Acad. Sci. U.S.A. 92:9363-9367; Chang et al., 1999, Oncogene 18:4808-4818). Cells were washed with phosphate buffered saline (PBS) supplemented with 1 mM MgCl₂, and then stained in X-gal solution (1 mg/ml X-gal, 0.12 mM K₃Fe[CN]₆, 0.12 mM K₄Fe[CN]₆, 1 mM MgCl₂ in PBS at pH 6.0) overnight at 37° C.

The senescence phenotypes were observed also in cells isolated from foci of Cdk4^{-/-} MEF expressing H-Ras^{Val12} and DNp53, and in cells isolated from foci of Cdk4−/− Ink4a/Arf^{-/-} MEF expressing H-Ras^{Val12}. These data suggested that the absence of Cdk4 induced senescence even with Ink4a/Arf disruption or p53 inhibition, which could account for the inhibition of oncogenic transformation.

### Example 5

### Cdk4-Null MEF Express High Levels of p21^{Cip1/Waf1} with Increased Stability

The mechanism of the resistance to Ras-mediated transformation in Cdk4-null cells was examined by determining expression of proteins that regulate senescence. In primary mouse and human cells, Ras activation or continuous passage in culture induces the expression of p15^{Ink4b}, p16^{Ink4a} and p21^{Cip1/Waf1}, as well as p19^{Arf} (or p 14^{Arf} in human cells) (Sherr and DePinho, 2000, Cell 102:407-410). Immunoblotting was used to determine expression of these proteins in Cdk4^{+/+} and Cdk4^{-/-} MEF. Cells were lysed by sonication in a tween-20-based lysis buffer, and 50 μg of proteins were analyzed by SDS-PAGE and western transfer, as described previously (Tsutsui et al., 1999, Mol. Cell Biol. 19:7011-7019). Antibodies were obtained from Neomarkers for Ras, Cdk6 and p16^{Ink4a}; from Santa Cruz Biotechnology for p15^{Ink4b} and p21^{Cip1/Waf1}; from Novus Biologicals for p19^{Arf}; from Sigma for actin. Immunoreactive bands were visualized using peroxidase-conjugated anti-Ig antibodies and the Supersignal chemiluminescence reagent (Pierce, Rockford, Ill.). Signals on X-ray films were quantified by using GS-700 Imaging Densitometer (Bio-Rad, Hercules, Calif.).

Cdk4^{+/+} and Cdk4^{-/-} MEF displayed similar induction of the expression of p15^{Ink4b}, p16^{Ink4a} and p19^{Arf} following H-Ras^{Val12} transduction (FIG. 6A). In contrast, the basal level of p21^{Cip1/Waf1} expression was significantly higher in Cdk4^{-/-} cells, relative to Cdk4^{+/+} cells, and H-Ras^{Val12} transduction increased p21^{Waf1/Cip1} expression even higher in Cdk4^{-/-} MEF. Similarly, Cdk4^{-/-} Ink4a/Arf^{-/-} MEF showed higher levels of p21^{Cip1/Waf1} than Cdk4^{+/+} Ink4a/Arf^{-/-} MEF. H-Ras^{Val12} did not significantly increase p21^{Waf1/CiP1} expression in cells with Ink4a/Arf disruption, which was consistent with the notion that p19^{Arf} played an essential role in stabilizing p53 and inducing p21^{Cip1/Waf1} upon Ras activation. H-Ras^{Val12} did not alter the expression of Cdk6 or p27^{Kip1}, regardless of the Cdk4 status.

To determine whether the increased basal levels of p21^{Waf1/Cip1}, in Cdk4-null cells were associated with p53 activity, the effect of DNp53 transduction on cellular expression of p21^{Cip1/Waf1} was examined (FIG. 6B). DNp53 significantly downregulated p21^{Cip1/Waf1} expression in both Cdk4^{+/+} and Cdk4^{-/-} MEF, confirming the role of p53 in p21^{Cip1/Waf1} transcription. In Cdk4^{-/-} MEF, which showed higher basal levels of p21^{Cip1/Waf1} expression, DNp53 transduction decreased p21^{Cip1/Waf1} only to a level comparable to the basal levels in Cdk4^{+/+} cells, suggesting that Cdk4 deficiency increased p21^{Cip1/Waf1} in a p53-independent manner.

To compare protein with mRNA levels, RT-PCR was used to analyze expression of p21^{Cip1/Waf1}, mRNA. RNA samples were prepared using the TRIZOL reagent (Life Technologies/Invitrogen). RT reactions were performed using the Superscript reverse transcriptase (Life Technologies/Invitrogen). The sequences of primers are:

| | |
|---|---|
| 5′-TGTCCAATCCTGGTGATGTCC3′ | (SEQ ID NO: 1) |
| and | |
| 5′-TCAGACACCAGAGTGCAAGAC-3′ | (SEQ ID NO: 2) |

for p21^{Cip1/Waf1};

| | |
|---|---|
| 5′-CCATCACTGCCACCCAGAAG-3′ | (SEQ ID NO: 3) |
| and | |
| 5′-TGGGTGCAGCGAACTTTATTG-3′ | (SEQ ID NO: 4) |

for GAPDH. PCR reactions were performed at 92° C. for 30 sec, 60° C. for 30 sec and 72° C. for 60 sec with 30 cycles, using the DNA Engine thermal cycler (MJ Research, Incline Village, Nev.). Semiquantitative conditions for the transcripts were worked out using increasing amounts of RNA. In contrast to the increased protein levels, the cellular amounts of p21^{Cip1/Waf1} mRNA were unchanged in Cdk4^{-/-} and Cdk4^{-/-} Ink4a/Arf^{-/-} MEF (FIG. 6C).

To examine degradation of p21^{Cip1/Waf1}, Cdk4^{-/-} MEF and Cdk4^{+/+} MEF were treated with the protein synthesis inhibitor cycloheximide (40 μg/ml) and cellular levels of p21^{Cip1/Waf1} were assayed by immunoblotting as described above. Three independent cell preparations at passage 3 or 4 for each genotype were examined. The results demonstrated that p21^{Cip1/Waf1} in Cdk4^{-/-} MEF was significantly more stable than in Cdk4^{+/+} MEF (FIG. 6D). Stability of p27^{Kip1} was also examined under these conditions, demonstrating that the degradation of p27^{Kip1} was similar in Cdk4^{-/-} and Cdk4^{+/+} MEF. These data suggested that Cdk4 deficiency resulted in a specific increase in p21^{Cip1/Waf1}, which could play a role in the senescence response.

Phosphorylation of Ser146, possibly by Akt (protein kinase-B) can stabilize p21 protein (Li et al., 2002, J. Biol. Chem. 277:11352-11361). To examine a possible mechanism of p21 stabilization in Cdk4-deficient cells, hemagglutinin (HA)-tagged wild-type or S146A mutant of human p21 constructs were prepared and expressed in Cdk4^{+/+} and Cdk4^{-/-} MEFs. Cycloheximide (CHX) treatment and chasing of exogenously expressed p21 by anti-HA immunoblotting showed that wild-type p21 showed increased stability in Cdk4^{-/-} MEFs, as expected (FIG. 7A). However, the S146A mutant was unstable in both and Cdk4^{+/+} and Cdk4^{-/-} MEFs, suggesting that phosphorylation at Ser141 (mouse counterpart of Ser146) may be increased in Cdk4-deficient MEFs and possibly involved in p21 stabilization. Immunoblotting with antibodies for S473^{P}Akt1 (activating phosphorylation of Akt1 at Ser473), Akt, Cdk4, and p³⁸MAPK showed that the expression of Akt proteins and activating phosphorylation of Akt1 at Ser473 were increased in two independent preparations of Cdk4^{-/-} MEFs, relative to wild-type control (FIG. 7B). Thus, Akt expression and activity may be increased in Cdk4-null cells, which could mediate p21 stabilization via Ser141 phosphorylation.

### Example 6

### Suppression of P21^{Cip1/Waf1} by siRNA Restores Immortalization and Ras-Mediated Transformation in Cdk4-Null MEF

To determine whether elevated p21^{Cip1/Waf1} expression in Cdk4-null MEF was required for the inhibition of immortalization and transformation, small interfering RNA (siRNA) were used to suppress cellular expression of p21^{Cip1/Waf1}. For suppression of cellular p21^{Cip1/Waf1} expression, siRNA that specifically targets p21^{Cip1/Waf1} mRNA was designed according to the manufacturer's protocol (Dharmacon Research, Lafayette, Colo.). The sense sequence was 5′-AACGGUGGAACUUUGACUUCG-3′ (SEQ ID NO: 5), corresponding to residues 136-156 of the coding region of mouse p21^{Cip1/Waf1} mRNA. MEF were transfected with the anti-p21^{Cip1/Waf1} siRNA or random 21-mer dsRNA (Dharmacon), using the Oligofectamine reagent (Life Technologies/Invitrogen, Rockville, Md.) according to the instruction of Dharmacon Research.

The 21-mer double stranded RNA was able to suppress cellular p21^{Cip1/Waf1} expression by more than 90%, suggesting a majority of cells were successfully transfected (FIG. 8A). The siRNA-based suppression of p21^{Cip1/Waf1} significantly restored clonogenic proliferation in low density-cultures of Cdk4^{-/-} Ink4a/Arf^{-/-} MEF (FIG. 8 B, C), suggesting that the elevated p21^{Cip1/Waf1} expression played a critical role in the limited proliferative life span.

Moreover, siRNA-mediated suppression of p21^{Cip1/Waf1} was able to restore foci formation significantly in Cdk4^{-/-} Ink4a/Arf^{-/-} cultures in response to H-Ras^{Val12} transduction (FIG. 8D). The numbers of Ras-induced foci in siRNA-treated Cdk4^{-/-} Ink4a/Arf^{-/-} cultures were about 75% of those in control Cdk4^{+/+} Ink4a/Arf^{-/-} cultures (24±3 vs 32±4, means ±SEM, n=3). The anti-p21^{Cip1/Waf1} siRNA treatment increased foci formation modestly (˜25%) in Cdk4^{+/+} Ink4a/Arf^{-/-} cultures. Transfection of the anti-p21^{Cip1/Waf1} siRNA also restored foci formation significantly in Cdk4^{-/-} MEF with transduction of H-Ras^{Val12} and DNp53. These data suggested that increased expression of p21^{Cip1/Waf1} by protein stabilization, which was independent of the Arf/p53 function, played an essential role in the resistance of Cdk4-null cells to immortalization and Ras-mediated transformation.

### Example 7

### The HPV7 Protein Fully Restores Transformation in Cdk4-Null MEF

The E7 oncoprotein of the human papillomavirus-16 (HPV) inactivates Rb by sequestration and destabilization (Dyson et al., 1989, Science 243:934-937; Boyer et al., 1996, Cancer Res. 56:4620-4624). E7 has also been shown to bind to the carboxyl terminus of p21^{Cip1/Waf1} and inactivate its Cdk-inhibitory and replication inhibitory actions (Funk et al., 1997, Genes Dev. 11:2090-2100). The HPV-E7 retrovirus packaging cell line, PA317 LXSN 16E7, was obtained from ATCC. E7 was expressed in Cdk4^{+/+} Ink4a/Arf^{-/-} and Cdk4^{-/-} Ink4a/Arf^{-/-} MEF by retroviral transduction as described above, followed by transduction of H-Ras^{Val12} or control vector, to determine whether the expression of E7 could restore the transformation potential in Cdk4-null cells (FIG. 9). The E7 retrovirus was used at maximum titers without dilution. Cdk4^{-/-} Ink4a/Arf^{-/-} MEF expressing H-Ras^{Val12} and E7 developed a number of transformed foci comparable to Cdk4^{+/+} Ink4a/Arf^{-/-} MEF expressing H-Ras^{Val12} with or without E7. Expression of E7 alone did not result in foci formation. The E7 retrovirus also restored foci formation in Cdk4^{-/-} MEF upon expression of H-Ras^{Val12} and DNp53 almost completely. These data indicated that the HPV E7 oncoprotein fully restored the transformation potential of Cdk4-disrupted cells.

### Example 8

### Premature Senescence Induced by siRNA-Based p53 or Cdk4 Silencing

To further examine the genetic interaction between Cdk4 and p53 in senescence control, a retrovirus carrying loop sequence for anti-Cdk4 siRNA was designed and constructed (FIG. 10). The sense sequence of the double-stranded siRNA was 5′-AAUCUACAGCUACCAGAUGGC-5′ (SEQ ID NO: 6), which corresponds to the nucleotide residue 489-509 of the mouse Cdk4 coding sequence. This sequence was chosen from 21-base sequences that include AA as initial two nucleotides and are highly specific to the Cdk4 gene in EST (expressed sequence tag) database, according to previous reports by Tom Tuschl's group (Elbashir et al., 2001, Genes Dev. 15:188-200; 2001, Nature 411:494-498). Tp53^{+/+} and Tp53^{-/-} MEFs were infected with the Cdk4 siRNA virus or control virus, and selected with puromycin for 72 h. Over 70% of cells were infected and showed puromycin resistance under the conditions. At 96 h postinfection, cells were examined by SA-β-gal staining as described above (FIG. 11A). Strikingly, almost 90% of Tp53^{-/-} MEFs exhibited SA-β-gal activity with flat and enlarged cytoplasm and ceased proliferation. In contrast, only a small fraction of Tp53^{+/+} MEFs showed SA-β-gal signals after Cdk4 silencing. DAPI staining and TUNEL assays performed as described in Zou et al., 2001, Mol. Cell. Biol. 21:4818-4828 and Jirawatnotai et al., 2003, J. Biol. Chem. 17021-17027, detected no obvious effect of anti-Cdk4 siRNA on cell death. The anti-Cdk4 siRNA decreased protein levels of Cdk4 by 80%, determined by immunoblotting with anti-Cdk4 antibodies at 96 h postinfection (FIG. 11B).

These data were consistent with the hypothesis that “immortal” proliferation of p53-deficient cells requires Cdk4. To test whether acute loss of p53 induced senescence response in Cdk4-deficient cells, a retrovirus for anti-p53 siRNA was generated as described in Dirac et al. (2003, J. Biol. Chem. 278:11731-11734), and Cdk4^{+/+} (WT) and Cdk4^{-/-} MEFs were infected with the virus, followed by puromycin selection. At 96-h postinfection, 90% of Cdk4^{-/-} MEFs displayed SA-β-gal activity (FIG. 11C). Proliferation of cultures infected with viruses concentrated by ultra-filtration were also examined (FIG. 11D). Cdk4^{+/+} MEFs with p53 siRNA transduction showed increased growth rates, suggesting that cells are undergoing immortalization. In contrast, Cdk4^{-/-} MEFs with p53 siRNA transduction barely proliferated, confirming their postmitotic state. These data were consistent with the lack of foci formation in Cdk4^{-/-} MEF cultures expressing H-Ras^{V12} and DNp53 as described above. Thus, loss of Cdk4 and p53 induced premature senescence in MEFs.

In addition, the Cdk4 siRNA was used to examine a panel of tumor cell lines with various p53 statuses. The Cdk4 siRNA was introduced into MCF7, MDA-MB-468, OVCAR-5 PA1, and A-2780 cells. Exponentially proliferating cells were infected with Cdk4 siRNA retrovirus, followed by selection with 2 mg/ml puromycin for 3 days. Cell proliferation was then assessed by the number and size of survived colonies. The results are shown in Table 2, which show that the Cdk4 siRNA had no effect on tumor cells having a wild type p53 gene, but inhibited growth of tumor cells having a mutated p53 gene.

TABLE 2
Impact of Cdk4 silencing on proliferation
of human cancer cell lines

| Cell line | Origin | p53 Status | Growth inhibition by Cdk4 siRNA |
|---|---|---|---|
| MCF7 | breast | WT | − |
| MDA-MB-468 | breast | Mutated | +++ |
| OVCAR-5 | ovarian | Mutated | +++ |
| PA1-I | ovarian | WT | − |
| A-2780 | Ovarian | WT | − |

It should be understood that the foregoing disclosure emphasizes certain specific embodiments of the invention and that all modifications or alternatives equivalent thereto are within the spirit and scope of the invention as set forth in the appended claims.

What is claimed is:

1. An siRNA inhibitor of Cdk4 activity, wherein the siRNA has a nucleotide sequence that corresponds to nucleotide residues in the mouse or human Cdk4 coding sequence and can inhibit growth of p53−/− cells.

2. The siRNA of claim 1, wherein the siRNA has a nucleotide sequence as shown in SEQ ID NO: 6.

3. A pharmaceutical composition comprising the siRNA of claim 1.

4. A method of treating a cancer patient comprising the step of administering the siRNA of claim 1 or the pharmaceutical composition of claim 3 to the patient.

5. The method of claim 4, wherein the cancer patient has a cancer that comprises tumor cells that are p53−/−.

6. The method of claim 4, wherein the cancer patient has a cancer that comprises tumor cells that comprise a mutant p53 gene.

7. The method of claim 4, wherein the cancer patient has a cancer that comprises tumor cells that comprise a mutant p53 protein.

8. A method of protecting a patient from developing cancer comprising the step of administering the pharmaceutical composition of claim 3 to the patient.

9. The method of claim 8, wherein certain of the patient's cells have a mutated p53 gene.

10. The method of claim 8, wherein certain of the patient's cells have a mutated p53 protein.

11. The method of claim 8, wherein certain of the patient's cells have a gene or protein in the p53 pathway that is mutated.

12. The method of claim 8, wherein the patient has an increased risk for developing cancer.

13. A method of inhibiting growth of a tumor cell comprising the step of contacting the cell with the siRNA of claim 1.

14. The method of claim 13, wherein the tumor cell is p53−/−.

15. The method of claim 13, wherein the tumor cell comprises a mutant p53 gene.

16. The method of claim 13, wherein the tumor cell comprises a mutant p53 protein.


SEQUENCE LISTING (6 sequences)

SEQ ID NO: 1 - DNA, 21 bp, Artificial
  PCR primer for p21
tgtccaatcctggtgatgtcc

SEQ ID NO: 2 - DNA, 21 bp, Artificial
  PCR primer for p21
tcagacaccagagtgcaagac

SEQ ID NO: 3 - DNA, 20 bp, Artificial
  PCR primer for GAPDH
ccatcactgccacccagaag

SEQ ID NO: 4 - DNA, 21 bp, Artificial
  PCR primer for GAPDH
tgggtgcagcgaactttattg

SEQ ID NO: 5 - RNA, 21 nt, Artificial
  siRNA for p21
aacgguggaacuuugacuucg

SEQ ID NO: 6 - RNA, 21 nt, Artificial
  Cdk4 siRNA
aaucuacagcuaccagauggc